(19) United States
(12) Patent Application Publication
(10) Pub. No.: US 2016/0287553 A1
(43) Pub. Date: Oct. 6, 2016

(54) TRANSLATION INHIBITORS IN HIGH-DOSE CHEMO- AND/OR HIGH-DOSE RADIOTHERAPY

(71) Applicant: DEUTSCHES KREBSFORSCHUNGSZENTRUM, Heidelberg (DE)

(72) Inventors: Michael S. BECKER, Nidderau (DE); Min LI-WEBER, Bad Durkheim (DE); Peter H. KRAMMER, Heidelberg (DE)

(21) Appl. No.: 15/038,236

(22) PCT Filed: Nov. 21, 2014
(86) PCT No.: PCT/EP2014/075230

§ 371 Date: May 20, 2016

(30) Foreign Application Priority Data
Nov. 22, 2013 (EP) 13194102.3

(51) Int. Cl. A61K 31/343; A61K 45/06

(52) U.S. Cl. CPC A61K 31/343; A61K 45/06

(57) ABSTRACT

The present invention relates to an inhibitor of protein translation for use in high-dose chemotherapy and/or high-dose radiotherapy of disease; to an inhibitor of protein translation for use in a combination therapy comprising high-dose chemotherapy and/or high-dose radiotherapy of disease; and to an inhibitor of protein translation for use in preventing adverse effects of high-dose chemotherapy and/or high-dose radiotherapy or for preventing radiation syndrome in a subject. Moreover, the present invention relates to a combined preparation for simultaneous, separate or sequential use comprising at least one inhibitor of protein translation or a pharmaceutically acceptable salt thereof; and at least one chemotherapeutic agent for use in high-dose chemotherapy of disease; to the use of an inhibitor of protein translation in high-dose chemotherapy and/or high-dose radiotherapy of disease; and to a medicament for the therapy of disease which contains (i) at least one inhibitor of protein translation or a pharmaceutically acceptable salt thereof, (ii) at least one chemotherapeutic agent, and (iii) at least one pharmaceutically acceptable carrier. Further, the present invention relates to a kit comprising at least one inhibitor of protein translation and instructions on administering high-dose chemotherapy and/or instructions on administering high-dose radiotherapy in the presence of said inhibitor of protein translation; as well as to improved methods of preventing in a subject requiring high-dose chemotherapy and/or high-dose radiotherapy adverse events caused by said therapy or therapies, of improving a medical condition requiring high-dose chemotherapy and/or high-dose radiotherapy; and of treating a subject in need of high-dose chemotherapy and/or high-dose radiotherapy.

The present invention relates to an inhibitor of protein translation for use in high-dose chemotherapy and/or high-dose radiotherapy of disease; to an inhibitor of protein translation for use in a combination therapy comprising high-dose chemotherapy and/or high-dose radiotherapy of disease; and to an inhibitor of protein translation for use in preventing adverse effects of high-dose chemotherapy and/or high-dose radiotherapy or for preventing radiation syndrome in a subject. Moreover, the present invention relates to a combined preparation for simultaneous, separate or sequential use comprising at least one inhibitor of protein translation or a pharmaceutically acceptable salt thereof; and at least one chemotherapeutic agent for use in high-dose chemotherapy of disease; to the use of an inhibitor of protein translation in high-dose chemotherapy and/or high-dose radiotherapy of disease; and to a medicament for the therapy of disease which contains (i) at least one inhibitor of protein translation or a pharmaceutically acceptable salt thereof, (ii) at least one chemotherapeutic agent, and (iii) at least one pharmaceutically acceptable carrier. Further, the present invention relates to a kit comprising at least one inhibitor of protein translation and instructions on administering high-dose chemotherapy and/or instructions on administering high-dose radiotherapy in the presence of said inhibitor of protein translation; as well as to improved methods of preventing in a subject requiring high-dose chemotherapy and/or high-dose radiotherapy adverse events caused by said therapy or therapies, of improving a medical condition requiring high-dose chemotherapy and/or high-dose radiotherapy; and of treating a subject in need of high-dose chemotherapy and/or high-dose radiotherapy.

Natural products are an important source of drugs in medicine. Recently, several studies showed that herbal extracts from traditional Chinese medicine (TCM) could reduce chemotherapy-induced side-effects in vivo (Goel et al., J Radiat Res. 2004; 45: 61-68; Mehendale et al., Am J Chin Med. 2004; 32: 897-905; Lee et al., Am J Chin Med. 1999; 27: 387-396; Lam et al., Sci Transl Med. 2010; 2: 45ra59). For instance, the herbal mixture PHY906, which is based on the TCM Huang Qin Tang, reduced CPT-11-induced toxicity in mice (Lam et al, op. cit.), a finding that is further supported by a phase 1/2 clinical trial (Farrell & Kummar, Clin Colorectal Cancer. 2003; 2: 253-256). Other clinical studies suggest that Chinese herbal extracts may reduce the chemotherapy-induced decrease in white blood cell counts (Chan et al., Ann Oncol. 2011; 22: 2241-2249).

Rocaglamide A (Roc-A) and its derivatives have been shown to possess anti-cancer activities in vitro in various tumor cell lines and patient samples and to inhibit tumor growth in vivo in several mouse tumor models (Kim et al., Anticancer Agents Med Chem. 2006; 6: 319-345; Ebada et al., Prog Chem Org Nat Prod. 2011; 94: 1-58). The primary effect of rocaglamides on tumor growth inhibition was shown to be due to inhibition of protein synthesis (Ohse et al., J Nat Prod. 1996; 59: 650-652; Lee et al., Chem Biol Interact. 1998; 115: 215-228). Two mechanisms, which ultimately lead to inactivation of the mRNA cap-binding eukaryotic translation initiation factor eIF4E and the translation initiation factor eIF4A, result in inhibition of protein synthesis (Polier et al., Chem Biol. 2012; 19: 1093-1104; Sadlish et al. ACS Chem Biol. 2013; doi:10.1021/cb400158t). It was proposed to use Rocaglamide derivatives as antineoplastic agents and in order to reduce cardiotoxicity and neurotoxicity of conventional antineoplastic therapy (WO 2010/060891, WO 2012/066002), as well as to use inducers of NFkappaB to prevent cells from undergoing apoptosis in cancer treatment (WO 2006/138238).

‘Classic’ genotoxic anti-cancer drugs all target DNA (Roos & Kaina, Cancer Lett. 2013; 332: 237-248). DNA damaging agents are potent inducers of cell death by triggering apoptosis not only in cancer but also in normal tissues. Especially, the toxicity to the hematopoietic system is the main challenge in anti-cancer treatment, as a decrease in white blood cell counts is usually the dose-limiting factor (Crawford et al., Cancer. 2004; 100: 228-237; Sinkule, Pharmacotherapy. 1984; 4: 61-73). Reduction in leukocytes causes weakening of the immune system and, thus, often leads to the development of opportunistic infections, which in the worst case can result in death of the patient (Mackall et al., Blood. 1994; 84: 2221-2228; Bodey et al., Anna Intern Med. 1966; 64: 328-340). Nevertheless, induction of DNA damage, such as DNA double-strand breaks (DSB), has been shown to be an effective treatment of cancer (Bonner et al., Nat Rev Cancer. 2008; 8: 957-967). In fact, most currently used anti-cancer drugs, e.g. Etoposide, Bleomycin, Doxorubicin, Teniposide, etc., act by causing DNA damage (ibid.). At present, the only drug approved by the FDA for improving side effects of cancer chemotherapy and radiotherapy is amifostine (2-(3-aminopropylamino)ethylsulfanyl phosphonic acid), which is believed to scavenge free radicals and other toxic metabolites.

Taken together, there is an urgent need for new therapeutic strategies which can reduce the toxicity of treatment on normal tissues but still maintain efficacy against the tumor. In particular, it is desirable to have means and methods at hand allowing to increase the dose of genotoxic agents and/or radiation tolerated by a patient, since this would allow for improved high-dose therapy increasing success rates in cancer treatment and/or improving quality of life of patients under treatment.

The problems as described above are solved by the means and methods provided by the present invention.

Accordingly, the present invention relates to an inhibitor of protein translation for use in high-dose chemotherapy and/or high-dose radiotherapy of disease.

As used in accordance with the present specification, the terms “treatment” and “therapy” relate to an amelioration of the diseases or disorders referred to herein or the symptoms accompanied therewith to a significant extent. Said treating as used herein also includes an entire restoration of the health with respect to the diseases or disorders referred to herein. It is to be understood that treating as used in accordance with the present invention may not be effective in all subjects to be treated. However, the term shall require that a statistically significant portion of subjects suffering from a disease or disorder referred to herein can be successfully treated. Whether a portion is statistically significant can be determined without further ado by the person skilled in the art using various well known statistic evaluation tools, e.g., determination of confidence intervals, p-value determination, Student's t-test, Mann-Whitney test etc. Preferred confidence intervals are at least 90%, at least 95%, at least 97%, at least 98% or at least 99%. The p-values are, preferably, 0.1, 0.05, 0.01, 0.005, or 0.0001. Preferably, the treatment shall be effective for at least 60%, at least 70%, at least 80%, or at least 90% of the subjects of a given cohort or population.

As used herein, the term “subject” relates to a vertebrate animal, preferably a mammal More preferably, the subject is a mouse, rat, hamster, guinea pig, cat, dog, sheep, cattle, horse, or pig. Most preferably, the subject is a human.

As used herein, the term “protein translation” relates to the process of decoding mRNA to produce an amino acid chain, i.e. a polypeptide, performed by ribosomes in eukaryotic cells. It is known to the skilled person that protein translation is generally divided into four steps, namely initiation, elongation, translocation and termination. It is understood by the skilled person that each of the aforesaid steps can be inhibited by appropriate chemical compounds as specified herein below.

The term “cell”, as used herein, relates to a living cell from a vertebrate animal, preferably a mammal More preferably, the cell is a cell of a mouse, rat, hamster, guinea pig, cat, dog, sheep, cattle, horse, or pig. Most preferably, the cell is a cell of a human Preferably, the cell is an isolated cell. More preferably, the cell is a cell comprised in a tissue, an organ, and/or a subject.

According to this specification, the term “inhibitor” relates to a chemical compound reducing the rate at which a specific process (the inhibited process) occurs or which prevents said process from progressing or from occurring. Thus, an “inhibitor of protein translation” is a compound reducing the rate at which protein translation occurs in the cell, or, preferably, preventing protein translation from progressing or from occurring. Preferably, the inhibitor of protein translation inhibits protein translation by inhibiting one of the macromolecules involved in protein biosynthesis, more preferably a macromolecule selected from the group consisting of initiation factors, mRNA, rRNA, ribosomal proteins, elongation factors, termination factors, and complexes formed between any two or more of these. More preferably, the inhibitor of protein translation inhibits protein translation by binding to one of the aforesaid macromolecules. Preferably, the inhibitor of protein translation inhibits protein translation by at least 25%, more preferably by at least 50%, still more preferably by at least 75%, or, most preferably, by at least 90%. Preferably, the inhibitor of protein translation is specific, i.e. specifically has the effect of inhibiting protein translation, more preferably without modulating cellular processes other than the ones described in the present specification to a detectable extent. Preferably, the inhibitor of protein translation inhibits protein translation when brought into contact with a cell. More preferably, the inhibitor of protein translation inhibits protein translation when provided in the medium surrounding a cell. Preferably, the inhibitor of protein translation is a reversible inhibitor of protein translation. More preferably, the reversible inhibitor of protein translation has a half-life in the body of a healthy subject of at most 30 days, more preferably of at most 15 days, even more preferably of at most 5 days, most preferably of at most 1 day. Preferably, the inhibitor of protein translation is an inhibitor of p53 translation.

Preferably, the inhibitor of protein translation is a didemnin B analogue such as Aplidin (Plitidepsin; CAS number: 137219-37-5), a cephalotaxus alkaloid such as Omacetaxine (Homoharringtonine, CAS number 26833-87-4), or a quassinoid, such as Bruceantin (CAS number 41451-75-6). More preferably, the inhibitor of protein translation is a flavagline.

The term “flavagline”, as used herein, relates to a chemical compound comprising a cyclopenta[b]benzofuran skeleton, preferably a cyclopenta[b]tetrahydroxy-benzofuran. As used in this specification, said terms include derivatives of the said compounds as described herein.

Preferably, the term flavagline relates to a compound of the formula (I)

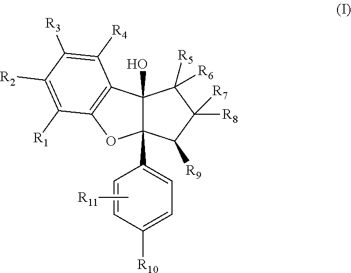

more preferably of the formula (X)

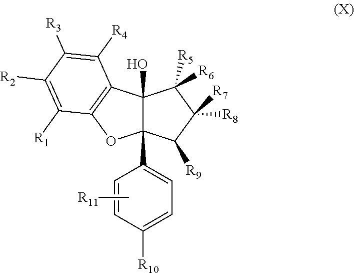

wherein
R₁ is selected from —H, halogen and alkyl;
R₂ is selected from alkoxy, optionally substituted, preferably selected from the group consisting of methoxy and a group —O—(CH₂)ₙ—R₁₈ wherein n is 1, 2, 3 or 4 and R₁₈ is hydroxyl, —NMe₂, —OCONMe₂, —OCONH₂ or morpholine, or R₂ is selected from halogen, and alkyl;
R₃ is selected from —H, halogen and alkyl;
   or R₂ and R₃ together form a —O(CH₂)ₙO— unit, with n=1 or 2;
R₄ is selected from alkoxy, halogen, —H, and alkyl;
R₅ is selected from hydroxyl, acyloxy, —H, amino, preferably substituted amino selected from the group consisting of monoalkylamino, dialkylamino, —NHCHO, —NHSO₂Me, —NHAc, —NHCOEt, —NHCOCH₂OH, —NHCOCH₂NMe₂, —NHCONMe₂, —NHCONH₂, and —NHCOOMe; and —NR₁₂—CHR₁₃—COOR₁₄, with
   R₁₂ being selected from —H and alkyl,
   R₁₃ being selected from phenyl and benzyl, which both may carry a substituent from the group hydroxyl, indolyl and imidazolylmethyl, and alkyl which may be substituted by a group selected from —OH, —SH, alkoxy, thioalkoxy, amino, monoalkylamino, dialkylamino, carboxy, carboxyalkyl, carboxamide and guanidino groups;
   or R₁₂ and R₁₃ together form a —(CH₂)₃— or —(CH₂)₄— group;
   R₁₄ being selected from alkyl and benzyl; in which case R₆ is hydrogen,
R₆ is selected from —H, halogen, alkyl, amino, preferably substituted amino selected from the group consisting of —NHCHO, —NHSO₂Me, —NHAc, —NHCOEt, —NHCOCH₂OH, —NHCOCH₂NMe₂, —NHCONMe₂, —NHCONH₂, and —NHCOOMe;
   or R₅ and R₆ together form an oxo or hydroxyimino group;
R₇ is —H;
R₈ is selected from —CONR₁₆R₁₇, —H, and —COOR₁₅ wherein
   R₁₅ and R₁₆ are independently selected from methyl and —H, and
   R₁₇ is selected from methyl, —H, 4-hydroxybutyl and 2-tetrahydrofuryl;
R₉ is selected from phenyl which is optionally substituted, preferably alkoxy- or halogen-substituted, and hetaryl which is optionally substituted;
R₁₀ is selected from alkoxy, —H, halogen, preferably —Br, and alkyl, and
R₁₁ is selected from —H, hydroxyl, halogen, alkoxy and alkyl;
   or R₁₀ and R₁₁ are in ortho-position to each other and together form a —O(CH₂)ₙO—unit, with n=1 or 2.

The term “alkyl”, as mentioned in the above definitions of the substituents R₁ to R₁₇, in each case refers to a substituted or an unsubstituted, linear or branched, acyclic or cyclic alkyl group, preferably an unsubstituted linear or branched acyclic alkyl group. More preferably, the term “alkyl”, as mentioned in the above definitions of the substituents R₁ to R₁₇, in each case preferably refers to a C₁- to C₄-alkyl group, namely methyl, ethyl, i-propyl, n-propyl, n-butyl, i-butyl, sec-butyl or tert-butyl. The above also applies when “alkyl” is used in “alkylamino” and “dialkylamino” and other terms containing the term “alkyl”.

The term “alkoxy”, as mentioned in the above definitions of the substituents R₁ to R₁₇, in each case refers to a substituted or an unsubstituted linear or branched, acyclic or cyclic alkoxy group, preferably an unsubstituted linear or branched acyclic alkoxy group. More preferably, the term “alkoxy”, as mentioned in the above definitions of the substituents R₁ to R₁₇, in each case preferably refers to a C₁- to C₄-alkoxy group, namely methoxy, ethoxy, i-propyloxy, n-propyloxy, n-butyloxy, i-butyloxy, sec-butyloxy or tert-butyloxy. The above also applies when “alkoxy” is used in “thioalkoxy” and other terms containing the term “alkoxy”.

The term “acyloxy”, as mentioned in the above definitions of the substituents R₁ to R₁₇, in each case refers to a substituted or an unsubstituted linear or branched, acyclic or cyclic acyloxy group, preferably an unsubstituted linear or branched acyclic acyloxy group. More preferably, the term “acyloxy”, as mentioned in the above definitions of the substituents R₁ to R₁₇, in each case preferably refers to a C₁- to C₄-acyloxy group, namely formyloxy, acetoxy, i-propyloxy, n-propyloxy, n-butyloxy, i-butyloxy, sec-butyloxy or tert-butyloxy.

The term “hetaryl” as used in the above definition refers to a 5-,6- or 7-membered carbocyclic saturated or non-saturated, aromatic or non-aromatic ring which may carry in the ring one or more heteroatoms from the group O, S, P, N.

The term “halogen” is known to the skilled person and preferably includes pseudhalogens; more preferably, the term relates to —F, —Cl, —Br, —I, —CN, or —SCN. Most preferably, the term relates to —Cl or —Br.

It is understood by the skilled person that formula (I) includes compounds wherein R₆ is orientated above the plane of view and R₅ then is orientated below the plane of view or vice versa. The same is true for R₇ and R₈ in formula (I), whereas in formula (X), R₅ and R₈ are orientated below the plane of view and R₆ and R₇ are orientated above the plane of view.

In a preferred embodiment of the present invention, the substituents R₁ to R₁₄ in formulae (I) and (X) have the following meanings:

R₁ and R₃ each are —H;
R₂ and R₄ each are independently selected from methoxy which is optionally substituted;
R₅ is selected from hydroxy, formyloxy and acetyloxy, alkylamino, —NR₁₂—CHR₁₃—COOR₁₄, with
R₁₂ being selected from —H and alkyl,
R₁₃ being selected from: alkyl which may be substituted by —OH, —SH, alkoxy; thioalkoxy, amino, alkylamino, carboxy, carboxyalkyl, carboxamide and/or guanidino groups; and phenyl and benzyl, which both may carry a substituent from the group hydroxy, indolyl and imidazolylmethyl;
R₁₄ being selected from alkyl and benzyl;

### R₆ is —H;

### R₇ is —H;

R₈ is selected from —H, —COOCH₃, and —CONR₁₆R₁₇, with R₁₆R₁₇ being independently selected from alkyl and cycloalkyl, which may be substituted, preferably —CON(CH₃)₂;
R₉ is phenyl which is optionally substituted;
R₁₀ is methoxy;
R₁₁ is selected from —H and hydroxy,
or R₁₀ and R₁₁ are in ortho-position to each other and together form a —O(CH₂)ₙO—unit, with n=1 or 2.

In a still more preferred embodiment of the present invention, the flavagline relates to those of formula (I) or formula (X), wherein

R₁ and R₃ each are —H,
R₂ and R₄ each are optionally substituted methoxy,
R₅ is hydroxy or —NR₁₂—CHR₁₃—COOR₁₄,
with R₁₂ being selected from —H and alkyl,
R₁₃ being selected from: alkyl which may be substituted by —OH, —SH, alkoxy; thioalkoxy, amino, alkylamino, carboxy, carboxyalkyl, carboxamide and/or guanidino groups; and phenyl and benzyl, which both may carry a substituent from the group hydroxy, indolyl and imidazolylmethyl;
R₁₄ being selected from alkyl and benzyl;
   R₆ and R₇ each are —H,
   R₈ is —CON(CH₃)₂,
   R₉ is optionally substituted phenyl,
   R₁₀ is methoxy and
   R₁₁ is —H; or wherein
   R₁ and R₃ each are —H,
   R₂ and R₄ each optionally substituted methoxy,
   R₅ is acetoxy or —NR₁₂—CHR₁₃—COOR₁₄,
   with R₁₂ being selected from —H and alkyl,
R₁₃ being selected from: alkyl which may be substituted by —OH, —SH, alkoxy; thioalkoxy, amino, alkylamino, carboxy, carboxyalkyl, carboxamide and/or guanidino groups; and phenyl and benzyl, which both may carry a substituent from the group hydroxy, indolyl and imidazolylmethyl;
R₁₄ being selected from alkyl and benzyl;
   R₆ and R₇ each are —H,
   R₈ is —CON(CH₃)₂,
   R₉ is optionally substituted phenyl,
   R₁₀ is methoxy and
   R₁₁ is —H; or wherein
   R₁ and R₃ each are —H,
   R₂ and R₄ each optionally substituted methoxy,
   R₅ is formyloxy or —NR₁₂—CHR₁₃—COOR₁₄,
   with R₁₂ being selected from —H and alkyl,
R₁₃ being selected from: alkyl which may be substituted by —OH, —SH, alkoxy; thioalkoxy, amino, monoalkylamino, dialkylamino, carboxy, carboxyalkyl, carboxamide and/or guanidino groups; and phenyl and benzyl, which both may carry a substituent from the group hydroxy, indolyl and imidazolylmethyl;
R₁₄ being selected from alkyl and benzyl;
   R₆ and R₇ each are —H,
   R₈ is —H or —COOCH₃,
   R₉ is optionally substituted phenyl, and
   R₁₀ and R₁₁ are in ortho-position to each other and together form a —O(CH₂)ₙO— unit, with n=1 or 2.

In a further embodiment of the present invention, R₈ is a group of the formula (c)

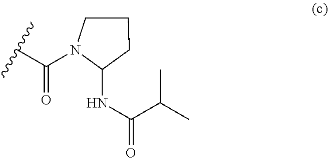 [SMILES: *C(=O)N1CCCC1NC(=O)C(C)C]

In still a further embodiment of the present invention, R₅ and R₈ together form a group of the formulae (a) or (b)

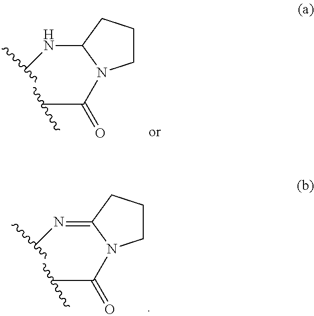 [SMILES: *NC1CCCN1C(*)=O ; "or" ; */N=C1/CCCN1C(*)=O]

Preferably, the term flavagline relates to a compound selected from the group consisting of rocaglamide, aglaroxin C, cyclorocaglamide, rocaglaol, methylrocaglate (aglafolin), desmethylrocaglamide, pannellin and the recently isolated dioxanyloxy-modified derivatives silvestrol and episilvestrol (Hwang et al., 2004, J. Org. Chem. Vol. 69: pages 3350-3358). It is understood by the skilled person that the term “rocaglamide”, preferably, is a generic term including compounds of formula (II) (named Rocaglamide A or Roc-A in the example section), formula (III) (named Rocaglamide AB), formula (IV), formula (V) (named Rocaglamide Q or Roc-Q in the example section), formula (VI) (referred to as Rocaglamide AR or Roc-AR in the present application), formula (VII) (known as Rocaglamide U or Roc-U), formula (VIII) (known as Rocaglamide W or Roc-W), or formula (IX) (known as Rocaglamide J). Preferably, the flavagline is not Rocaglamide AA (C-1-O-acetyl-methylrocaglate), Rocaglamide AF (30,40-methylendioxy-methylrocaglate) or Rocaglamide I (C-1-O-acetyl-30-hydroxy-rocaglamide). More preferably, the flavagline is Rocaglamide Q (demethylrocaglamide), Rocaglamide AR (1-oxo-40-demethoxy-30,40-methylenedioxyrocaglaol), Rocaglamide J (30-hydroxyaglafoline); even more preferably, the flavagline is Rocaglamide AB (1-O-acetyl-rocaglamide) or racemic bromo-demethoxy-rocaglaol (known as FL3 from WO 2010/060891); most preferably, the flavagline is Rocaglamide A ((1R,2R,3 S,3aR,8bS)-1,8b-dihydroxy-6,8-dimethoxy-3a-(4-methoxyphenyl)-N,N-dimethyl-3-phenyl-2,3-dihydro-1H-cyclopenta[b][1]benzofuran-2-carboxamide).

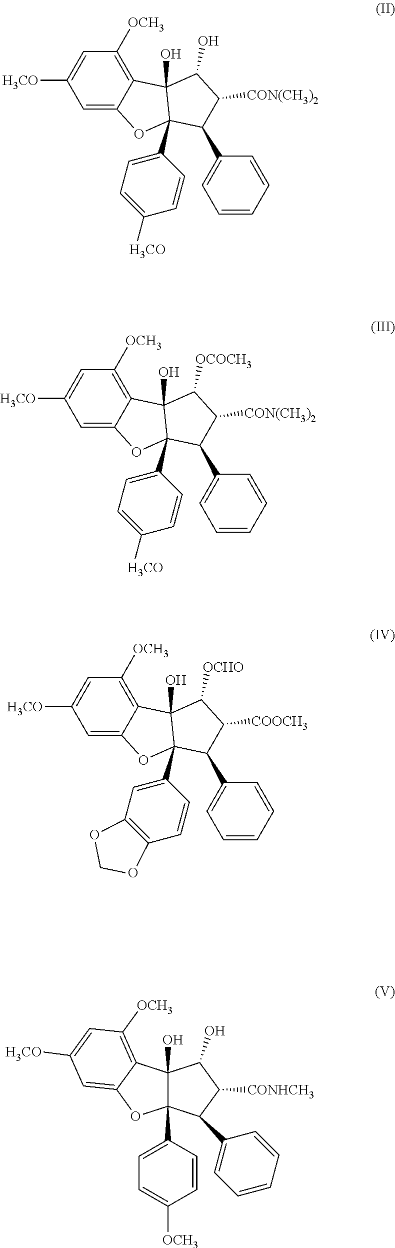

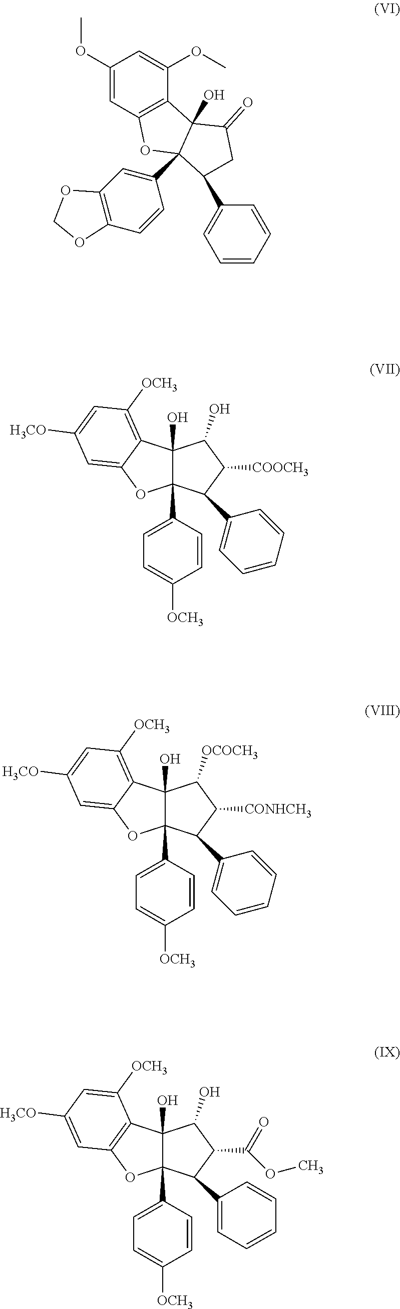

For the preparation of the rocaglamide derivatives according to the present invention, reference is made to WO 00/07579, WO 03/045375 and WO 00/08007.

Preferably, the term “inhibitor of protein translation” includes derivatives of the specific compounds described above and pharmaceutically acceptable salts of said compounds and derivatives.

The term “derivative”, as used herein, is known to the skilled person and relates to a compound obtainable from an active compound according to the present invention by chemical modification in, preferably, at most three chemical modification reactions, more preferably, in at most two chemical modification reactions, or, most preferably, in one chemical modification reaction. Preferably, the derivative comprises the same structural skeleton as the parent compound as described herein above and below. More preferably, the derivative has the same or a similar activity with regard to the diseases referred to herein as the parent compound as described herein above and below; or, also preferably, the derivative is an inactive precursor which is metabolized by the metabolism of the subject treated with said derivative into an active compound having the same or a similar activity with regard to the diseases referred to herein as the parent compound as described herein above and below. Preferred derivatives are compounds obtained from the compounds of the present invention by alkylation, preferably methylation or ethylation, acylation, preferably acetylation, glycosylation, hydroxylation, deacylation or demethylation, or derivatization with a piperazine, piperidine, piperidinamine, teneraic acid, piperidinepropanol, halogen, preferably F or Cl, more preferably I or Br, amino acid, or polypeptide, preferably olipopeptide, functional group.

As used herein, the term “pharmaceutically acceptable salt” refers to those salts which are, within the scope of sound medical judgment, suitable for use in contact with the tissues of humans and animals without undue toxicity, irritation, allergic response and the like, and are commensurate with a reasonable benefit/risk ratio. Pharmaceutically acceptable salts are well known in the art. For example, S. M. Berge, et al. describe pharmaceutically acceptable salts in detail in J. Pharmaceutical Sciences, 66: 1-19 (1977). The salts can be prepared in situ during the final isolation and purification of the inhibitor of protein translation or derivative, or separately by reacting the free base function with a suitable organic acid. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, arginine, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate.

As used herein, the term “chemotherapy” relates to treatment of a subject with an antineoplastic agent. Preferably, chemotherapy is a treatment including administration of an anaplastic lymphoma kinase (ALK)-inhibitor (e.g. Crizotinib or AP26130), an HDAC8-Inhibitor, an antiangiogenic agent (e.g. Bevacizumab), or an aurora kinase inhibitor (e.g. N-[4-[4-(4-Methylpiperazin-1-yl)-6-[(5-methyl-1H-pyrazol-3-yl)amino]pyrimidin-2-yl]sulfanylphenyl]cyclopropanecarboxamide (VX-680)). More preferably, chemotherapy is a treatment including administration of an antimetabolite (e.g. 5-fluorouracil, cytarabine, gemcitabine, fludarabine), a vinca alkaloid (e.g. vincristine, vinblastine), or a taxan (e.g. paclitaxel, docetaxel). Most preferably, chemotherapy is a treatment including administration of an alkylating agent (e.g. cyclophosphamide), a platinum compound (e.g. carboplatin), an antibiotic chemotherapeutic (e.g. bleomycin), an anthracycline (e.g. doxorubicin, epirubicin, idarubicin, or daunorubicin), or a topoisomerase II inhibitor (e.g. etoposide, irinotecan, teniposide, topotecan, camptothecin, or VP16), alone or any suitable combination thereof. Preferably, chemotherapy is a treatment including administration of at least one agent inducing DNA damage in a living cell.

The term “radiotherapy” (or “radiation therapy”), as used herein, relates to a treatment of a subject comprising administration of high-energy radiation. It is understood by the skilled person that the term includes all types of radiotherapy, including, but not limited to, external beam radiation therapy (e.g. X-ray therapy, particle therapy, or Auger therapy), brachytherapy (internal radiation therapy), and radioisotope therapy.

As used herein, the term “high-dose chemotherapy” relates to chemotherapy comprising administration of at least one chemotherapeutic agent at a dose higher than a standard dose of conventional chemotherapy as specified in guidelines of the guideline program of the Association of the Scientific Medical Societies AMWF, the German Cancer Society DKG and the German Cancer Aid DKH (“Leitlinienprogramm Onkologie der Arbeitsgemeinschaft der Wissenschaftlichen Medizinischen Fachgesellschaften e.V. (AWMF), der Deutschen Krebsgesellschaft e.V. (DKG) und der Deutschen Krebshilfe e.V. (DKH).”; Interdisziplinäre S3-Leitlinie für die Diagnostik und Therapie des Mammakarzinoms der Frau (2012), Stufe-3-Leitlinie zur Brustkrebsfrüherkennung (2008); Interdisziplinäre S2k-Leitlinie für die Diagnostik und Therapie des Endometriumkarzinom (2008); Interdisziplinäre S 2-Leitlinie für die Diagnostik und Therapie der Gliome des Erwachsenenalters; (2004) Interdisziplinäre Leitlinien zur Diagnostik und Behandlung von Hauttumoren (2005); S3-Leitlinie Hepatozelluläres Karzinom-HCC (2013); S3-Leitlinie Malignes Melanom (2013); 3-Leitlinie Diagnostik, Therapie und Nachsorge des Hodgkin Lymphoms bei erwachsenen Patienten (2013); Leitlinienkonferenz, “Kolorektales Karzinom” (2013); Interdisziplinäre S3-Leitlinie Prävention, Diagnostik, Therapie und Nachsorge des Lungenkarzinoms (2010); Interdisziplinäre S3-Leitlinie für die Diagnostik und Therapie der Adenokarzinome des Magens und ösophagogastralen Übergangs (2012); S3-Leitlinie “Diagnostik und Therapie des Mundhöhlenkarzinoms” (2012); S3-Leitlinie “S3-Leitlinie Diagnostik, Therapie und Nachsorge maligner Ovarialtumoren” (2013); S3-Leitlinie Exokrines Pankreaskarzinom (2006); S3-Leitlinie zur Früherkennung, Diagnose und Therapie des Prostatakarzinoms (2011); Interdisziplinäre S2k-Leitlinie für die Diagnostik und Therapie des Vulvakarzinoms und seiner Vorstufen (2009); Interdisziplinäre S2k-Leitlinie für die Diagnostik und Therapie des Zervixkarzinoms (2008)). Preferably, high-dose chemotherapy is chemotherapy comprising administation of at least one chemotherapeutic agent at a dose at least twice as high as a standard dose of conventional chemotherapy as specified in the guidelines recited above. Preferably, high-dose chemotherapy is a chemotherapy comprising administering a dose of at least one chemotherapeutic agent causing at least one grade 3 or higher adverse effect according to Common Toxicity Criteria (CTC) in at least 25% of patients receiving said dose. More preferably, the high-dose chemotherapy is a chemotherapy comprising administering a dose of at least one chemotherapeutic agent causing at least one grade 3 or higher adverse effect according to Common Toxicity Criteria (CTC) in at least 50% of patients receiving said dose. More preferably, high-dose chemotherapy is chemotherapy causing terminal failure of the bone marrow of the subject treated, i.e. a chemotherapy requiring a bone-marrow and/or stem cell transplant. Preferably, high-dose chemotherapy is chemotherapy comprising administering at least one compound/dose combination selected from the list consisting of: doxorubicin≧120 mg/m²/day, fludarabine≧350 mg/m²/day, ifosfamide≧10 g/m² (single dose), methotrexate≧500 mg/m² i.v., mitoxantrone≧30 mg, estramustine≧1120 mg/day, bleomycin≧30 U/m², vinblastine≧10 mg/m², docetaxol≧200 mg/m² i.v., thalidomide≧1000 mg/day, paclitaxel≧300 mg/m², tamoxifen≧60 mg/day, vinorelbine≧100 mg/m²/day, vincristine≧3 mg/m² day, dexamethazone≧60 mg/day, busulfan≧12 mg/kg/day, cyclophosphamide≧6000 mg/m², carmusine≧600 mg/m² i.v., cytosine arabinoside≧200 mg/m² day, thiotepa≧500 mg/m², carboplatin≧800 mg/m², etoposide≧625 mg/m² or ≧60 mg/kg, melphalan≧100 mg/m², mitoxantrone≧40 mg/m², cyclophosphamine≧100 mg/kg, and cyclophosphamine≧6 g/m². More preferably, high-dose chemotherapy is chemotherapy comprising high-dose administration of etoposide, bleomycin, doxorubicin, or teniposide as specified above.

The term “high-dose radiotherapy”, as used herein, relates to a radiotherapy comprising administration of at least one type of radiotherapy at a dose higher than a standard dose of conventional radiotherapy as specified in the guidelines of the guideline program of the Association of the Scientific Medical Societies AMWF, the German Cancer Society DKG and the German Cancer Aid DKH (“Leitlinienprogramm Onkologie der Arbeitsgemeinschaft der Wissenschaftlichen Medizinischen Fachgesellschaften e.V. (AWMF), der Deutschen Krebsgesellschaft e.V. (DKG) und der Deutschen Krebshilfe e.V. (DKH).”; Interdisziplinäre S3-Leitlinie fair die Diagnostik und Therapie des Mammakarzinoms der Frau (2012), Stufe-3-Leitlinie zur Brustkrebsfrüherkennung (2008); Interdisziplinäre S2k-Leitlinie für die Diagnostik und Therapie des Endometriumkarzinom (2008); Interdisziplinäre S 2-Leitlinie für die Diagnostik und Therapie der Gliome des Erwachsenenalters; (2004) Interdisziplinäre Leitlinien zur Diagnostik und Behandlung von Hauttumoren (2005); S3-Leitlinie Hepatozelluläres Karzinom-HCC (2013); S3-Leitlinie Malignes Melanom (2013); 3-Leitlinie Diagnostik, Therapie und Nachsorge des Hodgkin Lymphoms bei erwachsenen Patienten (2013); Leitlinienkonferenz “Kolorektales Karzinom” (2013); Interdisziplinäre S3-Leitlinie Prävention, Diagnostik, Therapie und Nachsorge des Lungenkarzinoms (2010); Interdisziplinäre S3-Leitlinie für die Diagnostik und Therapie der Adenokarzinome des Magens und ösophagogastralen Übergangs (2012); S3-Leitlinie “Diagnostik und Therapie des Mundhöhlenkarzinoms” (2012); S3-Leitlinie “S3-Leitlinie Diagnostik, Therapie und Nachsorge maligner Ovarialtumoren” (2013); S3-Leitlinie Exokrines Pankreaskarzinom (2006); S3-Leitlinie zur Früherkennung, Diagnose und Therapie des Prostatakarzinoms (2011); Interdisziplinäre S2k-Leitlinie fair die Diagnostik und Therapie des Vulvakarzinoms und seiner Vorstufen (2009); Interdisziplinäre S2k-Leitlinie für die Diagnostik und Therapie des Zervixkarzinoms (2008)); more preferably as specified in the guidelines of the German Society for Radiooncology DEGRO (“Leitlinien der Deutschen Gesellschaft für Radioonkologie (2013)”). Preferably, high-dose radiotherapy is radiotherapy comprising administation of a dose at least twice as high as a standard dose of conventional radiotherapy as specified in the guidelines recited above. Preferably, high-dose radiotherapy is a radiotherapy comprising administering a dose of radiation causing at least one grade 3 or higher adverse effect according to Common Toxicity Criteria (CTC) in at least 25% of patients receiving said dose. More preferably, the high-dose radiotherapy is a radiotherapy comprising administering a dose of radiation causing at least one grade 3 or higher adverse effect according to Common Toxicity Criteria (CTC) in at least 50% of patients receiving said dose. Most preferably, high-dose radiotherapy is radiotherapy causing terminal failure of the bone marrow of the subject treated, i.e. a radiotherapy requiring a bone-marrow and/or stem cell transplant.

The term “disease”, as used herein, relates to any disease or disorder which is known or expected to be cured or to show improvement after administration of high-dose chemotherapy and/or high-dose radiotherapy. Preferably, the disease is cancer. More preferably, the disease is a cancer selected from the list consisting of acute lymphoblastic leukemia, acute myeloid leukemia, adrenocortical carcinoma, aids-related lymphoma, anal cancer, appendix cancer, astrocytoma, atypical teratoid, basal cell carcinoma, bile duct cancer, bladder cancer, brain stem glioma, breast cancer, burkitt lymphoma, carcinoid tumor, cerebellar astrocytoma, cervical cancer, chordoma, chronic lymphocytic leukemia, chronic myelogenous leukemia, colon cancer, colorectal cancer, craniopharyngioma, endometrial cancer, ependymoblastoma, ependymoma, esophageal cancer, extracranial germ cell tumor, extragonadal germ cell tumor, extrahepatic bile duct cancer, gallbladder cancer, gastric cancer, gastrointestinal stromal tumor, gestational trophoblastic tumor, hairy cell leukemia, head and neck cancer, hepatocellular cancer, hodgkin lymphoma, hypopharyngeal cancer, hypothalamic and visual pathway glioma, intraocular melanoma, kaposi sarcoma, laryngeal cancer, medulloblastoma, medulloepithelioma, melanoma, merkel cell carcinoma, mesothelioma, mouth cancer, multiple endocrine neoplasia syndrome, multiple myeloma, mycosis fungoides, nasal cavity and paranasal sinus cancer, nasopharyngeal cancer, neuroblastoma, non-hodgkin lymphoma, non-small cell lung cancer, oral cancer, oropharyngeal cancer, osteosarcoma, ovarian cancer, ovarian epithelial cancer, ovarian germ cell tumor, ovarian low malignant potential tumor, pancreatic cancer, papillomatosis, paranasal sinus and nasal cavity cancer, parathyroid cancer, penile cancer, pharyngeal cancer, pheochromocytoma, pituitary tumor, pleuropulmonary blastoma, primary central nervous system lymphoma, prostate cancer, rectal cancer, renal cell cancer, retinoblastoma, rhabdomyosarcoma, salivary gland cancer, sézary syndrome, small cell lung cancer, small intestine cancer, soft tissue sarcoma, squamous cell carcinoma, squamous neck cancer, testicular cancer, throat cancer, thymic carcinoma, thymoma, thyroid cancer, urethral cancer, uterine sarcoma, vaginal cancer, vulvar cancer, waldenström macroglobulinemia, and wilms tumor. More preferably, the cancer is small cell lung cancer, a type of lymphoma, a type of leukemia. Preferably, the cancer is a cancer comprising or consisting of p53-deficient cancer cells; wherein p53-deficient cells are cancer cells not comprising the p53 activity as present in a normal cell, i.e., preferably, are cancer cells lower amounts of p53 as compared to normal cells and/or comprising a mutated p53 with a decreased propensity to be activated by cellular factors.

Advantageously, it was found in the work underlying the present invention that co-administration of an inhibitor of protein translation, in particular Rocaglamide, during high-dose chemotherapeutic treatment of cells protects primary cells, but not or to a lesser extent cancer cells, from entering apoptosis. A similar effect was identified in co-administration of an inhibitor of protein translation, in particular Rocaglamide, during high-dose radiation treatment of cells. This effect was most pronounced in cells of the blood system, in particular in hematopoietic stem and progenitor cells. Even more surprisingly, it was found that the same effect can be obtained by administering an inhibitor of protein translation after high-dose therapy. Accordingly, the means and methods of the present invention allow for a protection of non-cancer cells in high-dose therapy and, by reducing the rate and severity of adverse effects associated with high-dose therapy to more acceptable levels, make high-dose therapy possible at all in some therapeutic situations.

The definitions made above apply mutatis mutandis to the following. Additional definitions and explanations made further below also apply for all embodiments described in this specification mutatis mutandis.

The present invention also relates to a rocaglamide for use in high-dose chemotherapy and/or high-dose radiotherapy of disease.

The present invention further relates to an inhibitor of protein translation for use in a combination therapy comprising high-dose chemotherapy and/or high-dose radiotherapy of disease.

The term “combination therapy”, as used in this specification, relates to a treatment comprising administering the inhibitor of protein translation of the present invention and high-dose chemotherapy and/or high-dose radiotherapy to a subject. Preferably, the inhibitor of protein translation of the present invention is administered before high-dose chemotherapy and/or high-dose radiotherapy are administered. More preferably, the inhibitor of protein translation of the present invention and high-dose chemotherapy and/or high-dose radiotherapy are administered simultaneously, i.e. preferably, within a time frame of 48 hours, more preferably within a time frame of 24 hours.

The present invention also relates to an inhibitor of protein translation for use in preventing adverse effects of high-dose chemotherapy and/or high-dose radiotherapy or for preventing radiation syndrome in a subject.

The term “adverse effect”, as used herein, relates to a harmful and unintended effect resulting from the high-dose chemotherapy or the high-dose radiotherapy according to the present invention. Preferably, an adverse effect is a symptom or disorder correlating with loss of viable cells in fast-regenerating tissues or organs, e.g. indigestion, diarrhea, or malabsorption. More preferably, an adverse effect is a symptom or disorder caused by a distorted regeneration of blood cells (myelosuppression), e.g. thrombocytopenia, anemia, leukopenia (including neutropenia). Even more preferably, the adverse effect is a symptom or disorder caused by caused by a diminished number of T-cell, B-cells, NK cells, neutrophils and/or, most preferably, hematopoietic stem and progenitor cells.

The term “radiation syndrome” is known to the skilled person and relates to the specific combination of symptoms developed by subjects exposed to high doses of radiation, preferably ionizing radiation. Preferably, a high dose is a whole body absorbed dose of at least 0.25 Gy, more preferably of at least 0.5 Gy, even more preferably of at least 1 Gy, most preferably of at least 5 Gy. Preferably, a high dose is a whole body absorbed dose of less than 30 Gy, more preferably of less than 8 Gy, most preferably less than 6 Gy. Accordingly, a high dose of radiation, preferably, is a dose of 0.25 Gy to 30 Gy, more preferably of 0.5 Gy to 8 Gy, most preferably of 1 Gy to 6 Gy. Preferably, the symptoms of radiation syndrome prevented are nausea and diarrhea. More preferably, the symptoms of radiation syndrome prevented are leukopenia, purpura, hemorrhage, and infections.

The term “preventing” refers to retaining health or to diminishing the severity of at least one symptom with respect to the adverse effects or syndromes referred to herein for a certain period of time in a subject. It will be understood that the said period of time is dependent on the amount of the inhibitor of protein translation which has been administered and individual factors of the subject discussed elsewhere in this specification. It is to be understood that prevention may not be effective in all subjects treated with the compound according to the present invention. However, the term requires that a statistically significant portion of subjects of a cohort or population are effectively prevented from suffering from a disease or disorder referred to herein or its accompanying symptoms. Preferably, a cohort or population of subjects is envisaged in this context which normally, i.e. without preventive measures according to the present invention, would develop a disease or disorder as referred to herein. Whether a portion is statistically significant can be determined without further ado by the person skilled in the art using various well known statistic evaluation tools as described elsewhere herein.

Also, the present invention relates to a combined preparation for simultaneous, separate or sequential use comprising at least one inhibitor of protein translation or a pharmaceutically acceptable salt thereof; and at least one chemotherapeutic agent for use in high-dose chemotherapy of disease.

The term “combined preparation”, as used in this specification, relates to a preparation comprising the active compounds of the present invention for combined use. Thus, preferably, the combined preparation according to this specification is a preparation adapted such that the active compounds comprised therein are present in the body of a subject at an effective concentration for a certain time frame. More preferably, the active compounds are present in the body of a subject at an effective concentration sequentially or with overlapping time frames as described herein above. Preferably, the combined preparation is for simultaneous use, i.e., preferably, the combined preparation comprises the active compounds adjusted in dose and/or pharmaceutical form for combined use at the same time. More preferably, the combined preparation for simultaneous use comprises all pharmaceutically active compounds in one preparation so that all compounds are administered simultaneously and in the same way.

Also preferably, the combined preparation is for separate use, i.e., preferably, the combined preparation comprises at least two physically separated preparations for separate administration, wherein each preparation contains at least one pharmaceutically active compound. The embodiment comprising separate preparations is preferred in cases where the pharmaceutically active compounds of the combined preparation have to be administered by different routes, e.g. parenterally and orally, due to their chemical or physiological properties, or in cases where the active compounds are chemically incompatible. Preferably, the at least two separated preparations are administered simultaneously. This means that the time frames of the administration of the preparations overlap.

Also preferably, the combined preparation is for sequential use, i.e., preferably, the combined preparation is for sequential administration of at least two preparations, wherein each preparation contains at least one pharmaceutically active compound. In that case, the administration of the single preparations shall occur in time frames which do not overlap so that the at least two pharmaceutically active compounds of the preparations are present in such plasma concentrations which enable the synergistic therapeutic effect of the present invention. Preferably, the at least two preparations are administered in a time interval as described herein above. The embodiment of a preparation for sequential use is preferred in cases where the active compounds are of low physiological compatibility, e.g. because of an increase of adverse effects if taken simultaneously. Said embodiment is also preferred in cases where modes required modes of administration are temporally incompatible, e.g. in cases where one active compound is preferably administered before sleep, whereas the other is preferably administered in the morning.

The present invention further relates to a use of an inhibitor of protein translation in high-dose chemotherapy and/or high-dose radiotherapy of disease.

Moreover, the present invention relates to a medicament for the therapy of disease which contains (i) at least one inhibitor of protein translation or a pharmaceutically acceptable salt thereof, (ii) at least one chemotherapeutic agent, and (iii) at least one pharmaceutically acceptable carrier.

The term “medicament”, as used herein, relates to a pharmaceutical composition comprising or consisting of the active compounds of the present invention and optionally one or more pharmaceutically acceptable carrier. The active compounds of the present invention can be formulated as pharmaceutically acceptable salts as described herein above. The pharmaceutical compositions are, preferably, administered locally or topically, or, more preferably, systemically. Suitable routes of administration conventionally used for drug administration are oral, intravenous, or parenteral administration as well as inhalation. However, depending on the nature of an active compound and the disease to be treated, the pharmaceutical compositions may be administered by other routes as well. For example, peptides may be administered in a gene therapy approach by using viral vectors or viruses or liposomes.

Moreover, the active compounds can be administered in combination with other drugs either in a common pharmaceutical composition or as separated pharmaceutical compositions as described herein above. The active compounds are, preferably, administered in conventional dosage forms prepared by combining the drugs with standard pharmaceutical carriers according to conventional procedures. These procedures may involve mixing, granulating and compressing or dissolving the ingredients as appropriate to the desired preparation. It will be appreciated that the form and character of the pharmaceutically acceptable carrier or diluent is dictated by the amount of active ingredient with which it is to be combined, the route of administration and other well-known variables.

The carrier(s) must be acceptable in the sense of being compatible with the other ingredients of the formulation and being not deleterious to the recipient thereof. The pharmaceutical carrier employed may be, for example, either a solid, a gel or a liquid. Exemplary of solid carriers are lactose, terra alba, sucrose, talc, gelatin, agar, pectin, acacia, magnesium stearate, stearic acid and the like. Exemplary of liquid carriers are phosphate buffered saline solution, syrup, oil such as peanut oil and olive oil, water, emulsions, various types of wetting agents, sterile solutions and the like. Similarly, the carrier or diluent may include time delay material well known to the art, such as glyceryl mono-stearate or glyceryl distearate alone or with a wax. Said suitable carriers comprise those mentioned above and others well known in the art, see, e.g., Remington's Pharmaceutical Sciences, Mack Publishing Company, Easton, Pa.

The diluent(s) is/are selected so as not to affect the biological activity of the active compounds. Examples of such diluents are distilled water, physiological saline, Ringer's solutions, dextrose solution, and Hank's solution. In addition, the pharmaceutical composition or formulation may also include other carriers, adjuvants, or nontoxic, nontherapeutic, nonimmunogenic stabilizers and the like.

A therapeutically effective dose refers to an amount of the active compounds to be used in a pharmaceutical composition of the present invention, which prevents, ameliorates or treats the symptoms accompanying a disease or condition referred to in this specification. Therapeutic efficacy and toxicity of such active compounds can be determined by standard pharmaceutical procedures in cell cultures or experimental animals, e.g., ED50 (the dose therapeutically effective in 50% of the population) and LD50 (the dose lethal to 50% of the population). The dose ratio between therapeutic and toxic effects is the therapeutic index, and it can be expressed as the ratio, LD50/ED50.

The dosage regimen will be determined by the attending physician and other clinical factors; preferably in accordance with any one of the above-described methods. As is well known in the medical arts, dosages for any one patient depends upon many factors, including the patient's size, body surface area, age, the particular active compound to be administered, sex, time and route of administration, general health, and other drugs being administered concurrently. Progress can be monitored by periodic assessment. A typical dose can be, for example, in the range of 1 to 1000 μg; however, doses below or above this exemplary range are envisioned, especially considering the aforementioned factors. Generally, the regimen as a regular administration of the pharmaceutical composition should be in the range of 1 μg to 10 mg units per day. If the regimen is a continuous infusion, it should also be in the range of 1 μg to 10 mg units per kilogram of body weight per minute, respectively. Progress can be monitored by periodic assessment. However, depending on the subject and the mode of administration, the quantity of substance administration may vary over a wide range to provide from about 0.01 mg per kg body mass to about 10 mg per kg body mass, preferably.

The pharmaceutical compositions and formulations referred to herein are administered at least once in order to treat or ameliorate or prevent a disease or condition recited in this specification. However, the said pharmaceutical compositions may be administered more than one time, for example from one to four times daily up to a non-limited number of days.

Specific pharmaceutical compositions are prepared in a manner well known in the pharmaceutical art and comprise at least one active compound referred to herein above in admixture or otherwise associated with a pharmaceutically acceptable carrier or diluent. For making those specific pharmaceutical compositions, the active compound will usually be mixed with a carrier or the diluent, or enclosed or encapsulated in a capsule, sachet, cachet, paper or other suitable containers or vehicles. The resulting formulations are to be adapted to the mode of administration, i.e. in the forms of tablets, capsules, suppositories, solutions, suspensions or the like. Dosage recommendations shall be indicated in the prescribers or users instructions in order to anticipate dose adjustments depending on the considered recipient.

Moreover, the present invention relates to a kit comprising at least one inhibitor of protein translation and instructions on administering high-dose chemotherapy and/or instructions on administering high-dose radiotherapy in the presence of said inhibitor of protein translation.

The term “kit”, as used herein, refers to a collection of the aforementioned components, preferably, provided separately or within a single container. Examples for such components of the kit as well as methods for their use have been given in this specification. The kit, preferably, contains the aforementioned components in a ready-to-use formulation. The kit, preferably, additionally comprises a chemotherapeutic agent and/or a radiation source. Also preferably, the kit may comprise additional instructions, e.g., a user's manual or a package leaflet for administering the combined preparation or the medicament with respect to the applications provided by the methods of the present invention. Details are to be found elsewhere in this specification. Additionally, such user's manual may provide instructions about correctly using the components of the kit. A user's manual may be provided in paper or electronic form, e.g., stored on CD or CD ROM. The present invention also relates to the use of said kit in any of the methods according to the present invention. The kit of the present invention, preferably comprises a means for administering at least one of its components. The skilled person knows that the selection of the means for administering depends on the properties of the compound to be administered and the way of administration. Where the compound is or is comprised in a liquid and the mode of administration is oral, said means, preferably, is a drinking aid, such as a spoon or a cup. In case the liquid shall be administered intravenously, the means for administering may be an i.v. equipment.

The present invention also relates to a use of an inhibitor of protein translation for the manufacture of a medicament for treating and/or preventing adverse events in high-dose chemotherapy and/or high-dose radiotherapy and to a use of an inhibitor of protein translation for the manufacture of a combined medicament comprising said inhibitor of protein translation and a chemotherapeutic agent high-dose chemotherapy of disease.

Further, the present invention relates to a method of preventing in a subject requiring high-dose chemotherapy and/or high-dose radiotherapy adverse events caused by said therapy or therapies, comprising

a) administering an effective dose of an inhibitor of protein translation to said subject,
b) thereby preventing in a subject requiring high-dose chemotherapy and/or high-dose radiotherapy adverse events caused by said therapy or therapies.

The present invention also relates to a method of improving a medical condition requiring high-dose chemotherapy and/or high-dose radiotherapy, comprising

a) administering an inhibitor of protein translation to said subject,
b) administering high-dose chemotherapy and/or high-dose radiotherapy to said subject,
c) thereby improving a medical condition requiring high-dose chemotherapy and/or high-dose radiotherapy.

Moreover, the present invention relates to a method of treating a subject in need of high-dose chemotherapy and/or high-dose radiotherapy, comprising

a) administering a an inhibitor of protein translation to said subject,
b) administering high-dose chemotherapy and/or high-dose radiotherapy to said subject,
c) thereby treating cancer in a subject in need of high-dose chemotherapy and/or high-dose radiotherapy.

The methods of the present invention, preferably, are in vivo methods. Moreover, they may comprise steps in addition to those explicitly mentioned above. Also, one or more of said steps may be performed by automated equipment.

All references cited in this specification are herewith incorporated by reference with respect to their entire disclosure content and the disclosure content specifically mentioned in this specification.

Summarizing the findings of the present invention, the following embodiments are preferred:

### EMBODIMENT 1

An inhibitor of protein translation for use in high-dose chemotherapy and/or high-dose radiotherapy of disease.

### EMBODIMENT 2

An inhibitor of protein translation for use in a combination therapy comprising high-dose chemotherapy and/or high-dose radiotherapy of disease.

### EMBODIMENT 3

The inhibitor of protein translation for use of embodiment 1 or 2, wherein the disease is cancer.

### EMBODIMENT 4

The inhibitor of protein translation for use of any one of embodiments 1 to 3, wherein the high-dose chemotherapy is a chemotherapy comprising administering a dose of at least one chemotherapeutic agent causing at least one grade 3 or higher adverse effect according to Common Toxicity Criteria (CTC) in at least 50% of patients receiving said dose and/or wherein the high-dose radiotherapy is a radiotherapy comprising administering a dose of radiation causing at least one grade 3 or higher adverse effect according to CTC in at least 50% of patients receiving said dose.

### EMBODIMENT 5

The inhibitor of protein translation for use of any one of embodiments 1 to 4, wherein the inhibitor of protein translation is a flavagline, preferably of the formula (I)

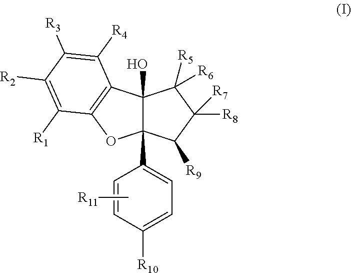

wherein
   R₁ is selected from —H, halogen and alkyl;
   R₂ is selected from alkoxy, optionally substituted, preferably selected from the group consisting of methoxy and a group —O—(CH₂)ₙ—R₁₈ wherein n is 1, 2, 3 or 4 and R₁₈ is hydroxyl, —NMe₂, —OCONMe₂, —OCONH₂ or morpholine, or R₂ is selected from halogen, and alkyl;
   R₃ is selected from —H, halogen and alkyl;
      or R₂ and R₃ together form a —O(CH₂)ₙO— unit, with n=1 or 2;
   R₄ is selected from alkoxy, halogen, —H, and alkyl;
   R₅ is selected from hydroxyl, acyloxy, —H, amino, preferably substituted amino selected from the group consisting of monoalkylamino, dialkylamino, —NHCHO, —NHSO₂Me, —NHAc, —NHCOEt, —NHCOCH₂OH, —NHCOCH₂NMe₂, —NHCONMe₂, —NHCONH₂, and —NHCOOMe; and —NR₁₂—CHR₁₃—COOR₁₄, with
      R₁₂ being selected from —H and alkyl,
      R₁₃ being selected from phenyl and benzyl, which both may carry a substituent from the group hydroxyl, indolyl and imidazolylmethyl, and alkyl which may be substituted by a group selected from —OH, —SH, alkoxy, thioalkoxy, amino, monoalkylamino, dialkylamino, carboxy, carboxyalkyl, carboxamide and guanidino groups;
      or R₁₂ and R₁₃ together form a —(CH₂)₃— or —(CH₂)₄— group;
      R₁₄ being selected from alkyl and benzyl; in which case R₆ is hydrogen,
   R₆ is selected from —H, halogen, alkyl, amino, preferably substituted amino selected from the group consisting of —NHCHO, —NHSO₂Me, —NHAc, —NHCOEt, —NHCOCH₂OH, —NHCOCH₂NMe₂, —NHCONMe₂, —NHCONH₂, and —NHCOOMe;
      or R5 and R6 together form an oxo or hydroxyimino group;
   R₇ is —H;
   R₈ is selected from —CONR₁₆R₁₇, —H, and —COOR₁₅ wherein
      R₁₅ and R₁₆ are independently selected from methyl and —H, and
      R₁₇ is selected from methyl, —H, 4-hydroxybutyl and 2-tetrahydrofuryl;
   R₉ is selected from phenyl which is optionally substituted, preferably alkoxy- or halogen-substituted, and hetaryl which is optionally substituted;
   R₁₀ is selected from alkoxy, —H, halogen, preferably —Br, and alkyl, and
   R₁₁ is selected from —H, hydroxyl, halogen, alkoxy and alkyl;
      or R₁₀ and R₁₁ are in ortho-position to each other and together form a —O(CH₂)ₙO—unit, with n=1 or 2.

### EMBODIMENT 6

The inhibitor of protein translation for use of any one of embodiments 1 to 5, wherein the inhibitor of protein translation is a Rocaglamide and wherein the inhibitor of protein translation is not Rocaglamide AA (C-1-O-acetyl-methylrocaglate), Rocaglamide AF (30,40-methylendioxy-methylrocaglate) or Rocaglamide I (C-1-O-acetyl-30-hydroxy-rocaglamide).

### EMBODIMENT 7

The inhibitor of protein translation for use of any one of embodiments 1 to 6, wherein the inhibitor of protein translation is Rocaglamide Q (demethylrocaglamide), Rocaglamide AR (1-oxo-40-demethoxy-30,40-methylenedioxyrocaglaol), Rocaglamide J (30-hydroxyaglafoline); preferably, is Rocaglamide AB (1-O-acetyl-rocaglamide) or racemic bromo-demethoxy-rocaglaol (FL3); more preferably, is (1R,2R,3S,3aR,8bS)-1,8b-dihydroxy-6,8-dimethoxy-3a-(4-methoxyphenyl)-N,N-dimethyl-3-phenyl-2,3-dihydro-1H-cyclopenta[b][1]benzofuran-2-carboxamide (Rocaglamide A; CAS number 84573-16-0) or a derivative thereof.

### EMBODIMENT 8

The inhibitor of protein translation for use of any one of embodiments 1 to 7, wherein high-dose chemotherapy is high dose therapy with an agent inducing DNA damage in cancer cells.

### EMBODIMENT 9

The inhibitor of protein translation for use of any one of embodiments 1 to 8, wherein high-dose chemotherapy is high dose therapy with an agent selected from the list consisting of etoposide, bleomycin, doxorubicin, teniposide.

### EMBODIMENT 10

An inhibitor of protein translation for use in preventing adverse effects of high-dose chemotherapy and/or high-dose radiotherapy or for preventing radiation syndrome in a subject.

### EMBODIMENT 11

The inhibitor of protein translation for use of embodiment 10, wherein the adverse effects are not neuronal and/or cardiac adverse effects.

### EMBODIMENT 12

The inhibitor of protein translation for use of embodiment 10 or 11, wherein the adverse effects are adverse effects of the blood system.

### EMBODIMENT 13

The inhibitor of protein translation for use of any one of embodiments 10 to 12, wherein the adverse effects are adverse effects caused by a diminished number of at least one kind of blood cell.

### EMBODIMENT 14

The inhibitor of protein translation for use of any one of embodiments 10 to 13, wherein the adverse effects are adverse effects caused by a diminished number of T-cells, B-cells, NK cells, neutrophils and/or hematopoietic stem and progenitor cells.

### EMBODIMENT 15

A combined preparation for simultaneous, separate or sequential use comprising at least one inhibitor of protein translation or a pharmaceutically acceptable salt thereof and at least one chemotherapeutic agent for use in high-dose chemotherapy of disease.

### EMBODIMENT 16

Use of an inhibitor of protein translation in high-dose chemotherapy and/or high-dose radiotherapy of disease.

### EMBODIMENT 17

A medicament for the therapy of disease which contains (i) at least one inhibitor of protein translation or a pharmaceutically acceptable salt thereof, (ii) at least one chemotherapeutic agent, and (iii) at least one pharmaceutically acceptable carrier.

### EMBODIMENT 18

A kit comprising at least one inhibitor of protein translation and instructions on administering high-dose chemotherapy and/or instructions on administering high-dose radiotherapy in the presence of said flavagline.

### EMBODIMENT 19

Use of an inhibitor of protein translation for the manufacture of a medicament for treating and/or preventing adverse effects in high-dose chemotherapy and/or high-dose radiotherapy.

### EMBODIMENT 20

Use of an inhibitor of protein translation for the manufacture of a combined medicament comprising said inhibitor of protein translation and a chemotherapeutic agent high-dose chemotherapy of disease.

### EMBODIMENT 21

A flavagline for use in high-dose chemotherapy and/or high-dose radiotherapy of disease or for preventing radiation syndrome in a subject.

### EMBODIMENT 22

A method of preventing in a subject requiring high-dose chemotherapy and/or high-dose radiotherapy adverse events caused by said therapy or therapies, comprising
a) administering an effective dose of an inhibitor of protein translation to said subject,
b) thereby preventing in a subject requiring high-dose chemotherapy and/or high-dose radiotherapy adverse events caused by said therapy or therapies.

### EMBODIMENT 23

A method of improving a medical condition requiring high-dose chemotherapy and/or high-dose radiotherapy, comprising
a) administering an inhibitor of protein translation to said subject,
b) administering high-dose chemotherapy and/or high-dose radiotherapy to said subject,
c) thereby improving a medical condition requiring high-dose chemotherapy and/or high-dose radiotherapy.

### EMBODIMENT 24

A method of treating a subject in need of high-dose chemotherapy and/or high-dose radiotherapy, comprising
a) administering an inhibitor of protein translation to said subject,
b) administering high-dose chemotherapy and/or high-dose radiotherapy to said subject,
c) thereby treating cancer in a subject in need of high-dose chemotherapy and/or high-dose radiotherapy.

### FIGURE LEGENDS

FIG. 1. Roc-A protects non-malignant cells from DNA damage-induced cytotoxicity

(a) Roc-A protects T cells from Etoposide-induced apoptotic cell death in a dose- and time-dependent manner Left panel: T cells were treated with solvent (DMSO) or increasing amounts of Etoposide in the absence or presence of different concentrations of Roc-A for 24 h. Apoptotic cell death was determined by DNA fragmentation. Data are an average of three independent experiments. Error bars (s.d.) are shown, middle panel: T cells were treated with 50 μM Etoposide in the absence (DMSO) or presence of different concentrations of Roc-A for indicated time-periods. Apoptotic cell death was determined by DNA fragmentation. Data are an average of three independent experiments. Error bars (s.e.m.) are shown, right panel: Roc-A was added 2 h prior, in parallel or 2 and 4.5 h after Etoposide (50 μM) treatment. Data are presented as percent of protection of T cells from Etoposide-induced apoptosis. Results are an average of three independent experiments. Error bars (s.e.m.) are shown. (b) Roc-A reduces Teniposide-, Doxorubicin- and Bleomycin-induced apoptotic cell death in T cells. Peripheral blood T cells were treated with Teniposide (left panel), Doxorubicin (middle panel) or Bleomycin (right panel) in the absence (DMSO) or presence of Roc-A (75 nM) as indicated. Apoptotic cell death was determined by DNA fragmentation for Teniposide and Bleomycin treatment or by FSC/SSC profile for Doxorubicin treatment. Data are an average of three independent experiments. Error bars (s.d.) are shown. (c) Roc-A protects a panel of non-transformed primary cells from Etoposide-induced cell death. Primary human B cells, NK cells, neutrophils, HSPCs and cardiomyocytes were treated with Etoposide in the absence (DMSO) or presence of different concentrations of Roc-A for different times as indicated. Cell death was determined by DNA fragmentation. The results are averages of three to four independent experiments. Error bars (s.d.) are shown. (d) Roc-A was added 1 h prior, or 1 h-4 h after IR treatment (10 Gy) of primary human T cells. Data are presented as percent of protection of T cells from IR-induced apoptosis. Results are an average of four independent experiments. Error bars (s.e.m.) are shown. (e) Left panel:Primary human T cells were treated with solvent (DMSO) or increasing doses of IR in the presence of different concentrations of Roc-A or solvent (DMSO) as indicated for 24 h. Apoptotic cell death was determined by DNA fragmentation (left panel) or FSC/SSC profile (right panel). Data are an average of three independent experiments. Error bars (s.d.) are shown.

FIG. 2. Roc-A does not protect T cells from genotoxin-induced DNA damage

(a) Roc-A does not prevent Etoposide-induced increase in γ-H2AX. T cells were treated with different concentrations of Etoposide without (DMSO) or with Roc-A (75 nM) for 4 h. DSB induction was assessed by determination of the mean fluorescence intensity (MFI) of γ-H2AX-stained living cells. Data are an average of three independent experiments. Error bars (s.d.) are shown. (b) Kinetic analysis of the effect of Roc-A on Etoposide-induced DSBs. T cells were treated with 50 μM Etoposide in the absence (DMSO) or presence of Roc-A (75 nM) for different times and DSB induction was determined as described in (a). Data are an average of three independent experiments. Error bars (s.d.) are shown. (c) Summary of the data obtained from a comet assay to monitor the effect of Roc-A on Etoposide-induced DNA damage in T cells. Peripheral blood T cells were treated with Etoposide (50 μM) in the absence (DMSO) or presence of Roc-A (75 nM) for different time periods as indicated. A comet assay was carried out subsequently. Results are an average of the mean olive tail moments (OTM) of three different healthy donors. Error bars (s.d.) are shown. *p<0.05, calculated by the unpaired Student's t-test with Welch's correction.

FIG. 3. Roc-A blocks genotoxin-induced upregulation of p53

(a and b) Roc-A inhibits Etoposide-(a), Bleomycin-, Teniposide- and Doxorubicin-(b) induced p53 upregulation in T cells. T cells were treated with different anti-cancer drugs in the presence or absence (DMSO) of different concentrations of Roc-A as indicated. Cell lysates were subjected to immunoblot analysis with antibodies against p53. Actin or tubulin were used as loading controls. Data are representative of three independent experiments. (c) Kinetic analysis of the effect of Roc-A on Etoposide-induced p53 upregulation. T cells were treated with 50 μM Etoposide and 75 nM Roc-A for different time periods as indicated. Cell lysates were subjected to immunoblot analysis with antibodies against p53 and tubulin. Data are representative of two independent experiments. (d and e) Roc-A inhibits Etoposide-induced p53 upregulation in B cells (d) and NK cells (e). Cells were treated with Etoposide and Roc-A as indicated and cell lysates were subjected to immunoblot analysis. Data are representative of two independent experiments. (f) Primary human T cells were pre-treated with solvent (DMSO) or 75 nM Roc-A for 1 h and subsequently exposed to 10 Gy IR or not exposed to IR as indicated. 24 h after exposure, cell lysates were subjected to immunoblot analysis with antibodies against p53. Tubulin was used as loading control. Data are representative of three independent experiments.

FIG. 4. Roc-A-mediated chemo-protection depends on p53

(a) siRNA-mediated knock-down of p53 mimics the protective effect of Roc-A. T cells were transfected with scrambled (si-Ctrl.) or specific siRNA against p53 (si-p53). 24 h after transfection, T cells were treated with Etoposide (50 μM) in the absence or presence of Roc-A (75 nM) as indicated for 24 h. p53 expression levels were analyzed by immunoblot and cell death was determined by FSC/SSC profile. Data are representative of three independent experiments. (b) Roc-A-mediated protection is abolished in p53^{-/-} splenocytes. Splenocytes from p53^{-/-} or p53^{+/+} mice were treated with 50 μM Etoposide in the absence or presence of 75 nM Roc-A for indicated time periods. Cell death was determined by DNA fragmentation. Data are an average of four independent experiments. Error bars (s.d.) are shown. Asterisks indicate statistical significance with **p<0.01, ****p<0.0001 calculated by unpaired Student's t-test with Welch's correction. Differences between DMSO- and Roc-A-treated p53KO cells were not statistically significant.

FIG. 5. Roc-A does not protect cancer cell lines with non-functional p53.

p53 mutated or deficient cancer cell lines (a) and p53 WT cell lines (b) were treated with different concentrations of Etoposide in the absence or presence of increasing amounts of Roc-A as indicated. Apoptotic cell death was determined by DNA fragmentation after 24 h or 48 h treatment as indicated. Results are averages of three independent experiments. Error bars (s.d.) are shown.

FIG. 6. Roc-A inhibits upregulation of p53 via inhibition of protein synthesis.

(a) Inhibition of proteasome-mediated degradation does not influence Roc-A-mediated chemo-protection. T cells were treated with 100 nM Bortezomib to block proteasome-mediated protein degradation and treated with 50 μM Etoposide in the absence or presence of 75 nM Roc-A for 4 h. Cell lysates were subjected to immunoblot analysis with antibodies against p53 and Actin. Data are representative of three independent experiments. (b) Inhibition of protein translation by Roc-A or its derivatives correlates with their chemo-protective effects. Effects of Roc-A and its derivatives (-AB, -J, -AR, -Q, —I, -AF, -AA) on protein synthesis in T cells was determined by measuring the amounts of incorporation of [³⁵S]-labeled methionine. Apoptotic cell death was determined by DNA fragmentation of T cells treated with 50 μM Etoposide in the absence or presence of 75 nM of different Roc-derivatives for 24 h. The percentage of chemo-protection was determined by calculating the percentage of protection against Etoposide-induced cell death. The data are shown by plotting the percentage of translation inhibition against the percentage of chemo-protection. Data are an average of three independent experiments. Error bars (s.e.m.) are shown. An allosteric sigmoidal regression curve was plotted against the experimental data. R²=0.96. (c) Roc-A inhibits p53 protein translation. T cells were treated according to (a), followed by metabolic pulse-labeling for indicated time periods and immunoprecipitation. Data are representative of three independent experiments.

FIG. 7. Roc-A reduces Etoposide-induced apoptosis in T cells.

T cells were treated with Etoposide and Roc-A as indicated for 24 h. Apoptosis was measured by FSC/SSC profile (left panel) or staining for AnnexinV (right panel). Data are an average of three independent experiments. Error bars (s.d.) are shown.

FIG. 8. FL3 protects T cells from ionizing radiation (IR)-induced and Etoposide-induced apoptotic cell death in a dose-dependent manner.

(a) Primary human T cells were treated with solvent (DMSO) or increasing doses of Etoposide in the presence of different concentrations of FL3 or solvent (DMSO) as indicated for 24 h. Apoptotic cell death was determined by DNA fragmentation. Data are an average of three independent experiments. Error bars (s.d.) are shown. (b) Primary human T cells were treated with solvent (DMSO) or increasing doses of IR in the presence of different concentrations of FL3 or solvent (DMSO) as indicated for 24 h. Apoptotic cell death was determined by DNA fragmentation. Data are an average of two independent experiments.

FIG. 9. Roc-A enables the use of high-dose chemo/radiotherapy by protecting healthy cells from DNA-damage induced cell death.

(a-b) Malignant and non-malignant cells were treated with Etoposide in the presence of 75 nM Roc-A or solvent (DMSO) and cell death was determined after 24 h by DNA fragmentation. Depicted is the fold change in cell death that was measured when doses of Etoposide were increased from 6.25 μM to 50 μM. (a) Cells from (b) were grouped into malignant and non-malignant cells. Shown are means of three independent experiments. Abbreviations; HSPCs=hematopoietic stem and progenitor cells. (c-d) Malignant and non-malignant cells were exposed to ionizing radiation (IR) in the presence of 75 nM Roc-A or solvent (DMSO) and cell death was determined after 24 h by DNA fragmentation. Depicted is the fold-change in cell death that was measured when doses of IR were increased from 2 Gy to 10 Gy. (c) Cells from (d) were grouped into malignant and non-malignant cells. Shown are means of three independent experiments.

FIG. 10. Translation Inhibitors protect T cells from Ionizing Radiation (IR)-induced apoptotic cell death.

T cells were pretreated with 100 nM Roc-A, 10 nM Bruceantin, 250 nM Didemnin B or 250 nM Omacetaxine for 1 h followed by exposure to 10 Gy IR. Unexposed T cells were used as controls. Apoptotic cell death was determined after 24 h by DNA fragmentation. Data are presented as percent of protection of T cells from IR-induced apoptosis. Data are an average of two independent experiments.

FIG. 11. Translation Inhibitors protect T cells from Etoposide-induced apoptotic cell death.

T cells were exposed to solvent (DMSO) or 50 μM Etoposide in the absence or presence of 100 nM Roc-A, 10 nM Bruceantin, 250 nM Didemnin B or 250 nM Omacetaxine for 24 h. Apoptotic cell death was determined by DNA fragmentation. Data are presented as percent of protection of T cells from Etoposide-induced apoptosis. Data are an average of two independent experiments.

The following Examples shall merely illustrate the invention. They shall not be construed, whatsoever, to limit the scope of the invention.

### EXAMPLE 1

### Materials and Methods

### Reagents and Roc-Derivatives

Etoposide (Biotrend Chemikalien GmbH, Köln, Germany), Bleomycin (sulfate) (Cayman Chemical Company, Michigan, USA), Doxorubicin (Sigma-Aldrich, Munich, Germany), and Teniposide (Enzo Life Sciences, Lörrach, Germany) were used for apoptosis induction. Roc-A (>98% pure) (Enzo Life Sciences, Lörrach, Germany) and derivatives Roc-AA (C-1-O-acetyl-methylrocaglate), Roc-AB (1-O-acetyl-rocaglamide), Roc-AF (30,40-methylendio xy-methylro-caglate), Roc-AR (1-oxo-40-demethoxy-30,40-methylenedioxyrocaglaol), Roc-I (C-1-O-acetyl-30-hydroxy-rocaglamide), Roc-J (30-hydroxyaglafoline) and Roc-Q (demethylrocaglamide) were isolated from Aglaia species to the purity >98% as determined by high-performance liquid chromatography (HPLC).

### Primary Human Cells and Cell Cultures

The human malignant cell lines EU-3 (acute lymphoblastic leukemia), DND-41 (T cell leukemia), Hut-78 (T cell lymphoma), SKW6.4 (B cell leukemia), Reh (acute lymphoblastic leukemia), IM-9 (Chronic myeloid leukemia), HL-60 (promyelocytic leukemia), L1236 (Hodgkin's lymphoma) and NCI-H209 (small cell lung cancer) were cultured at 37° C. with 5% CO₂ in RPMI-1640 medium (Sigma-Aldrich, Munich, Germany) supplemented with 10% FCS, 100 U/ml Penicillin (Sigma-Aldrich, Munich, Germany) and 100 μg/ml Streptamycin (Sigma-Aldrich, Munich, Germany) SCLC-21H cells (small cell lung cancer) were cultured in DMEM medium (Sigma-Aldrich, Munich, Germany) supplemented with 10% FCS. Peripheral blood T lymphocytes were isolated as previously described (Klas et al., Int Immunol. 1993; 5: 625-630). B lymphocytes and NK cells were isolated by magnetic activated cell sorting using “B cell isolation kit II” (Miltenyi Biotech, Bergisch Gladbach, Germany) and “NK cell isolation kit, human” (Miltenyi Biotech, Bergisch Gladbach, Germany), respectively, according to the manufacturer's instructions. Human neutrophils were separated from peripheral blood mononuclear cells by Ficoll-Paque density centrifugation, followed by incubation in 1.05% dextran for 30 min at room temperature. Remaining erythrocytes were lysed by resuspension in ice-cold 0.2% sodium chloride solution. After 1 min ice-cold 1.6% sodium-chloride solution was added and lysis was stopped by addition of PBS and neutrophils were resuspended in medium at a concentration of 2×10⁶ cells/ml. Human primary cardiomyocytes were purchased from PromoCell (Heidelberg, Germany) and cultured in Myocyte Growth Medium (PromoCell, Heidelberg, Germany). Remaining primary human cells were cultured in RPMI-1640 medium with the same conditions described above.

### Apoptosis Measurements

Apoptotic cell death was determined by AnnexinV staining, cellular forward scatter/side scatter (FSC/SSC) profile, or DNA fragmentation. For AnnexinV staining, 2×10⁵ cells were treated with different drugs for indicated time periods, washed with AnnexinV binding buffer (0.01 M Hepes, 0.14 M NaCl, 2.5 mM CaCl₂), and stained with AnnexinV-FITC antibody (Immunotools, Friesoythe, Germany) and 7-amino-actinomycin D (Sigma-Aldrich, Munich, Germany) for 30 min at 4° C. The amount of AnnexinV positive cells was determined by FACS measurement. DNA fragmentation was determined according to the method of Nicoletti (Nicoletti et al., J Immunol Methods. 1991; 139: 271-279). Briefly, 2×10⁵ cells were treated as indicated, washed with PBS and lysed in Nicoletti buffer (0.1% sodium citrate, 0.1% Triton X-100, 50 μg/ml propidium iodide). DNA fragmentation was determined by FACS. Apoptosis-like cells were determined by forward scatter and side scatter (FSC/SSC) index. Specific DNA fragmentation/specific AnnexinV positive cells/specific apoptosis was calculated as follows: (percentage of experimental DNA fragmentation (or Annexin V positive cells or apoptosis)−percentage of spontaneous DNA fragmentation (or Annexin V positive cells or apoptosis)/(100−percentage of spontaneous DNA fragmentation (or Annexin V positive cells or apoptosis))×100.

### Immunoblot Analysis

Immunoblot analysis was carried out as previously described (Polier et al., Chem Biol. 2012; 19: 1093-1104). Briefly, 4-20×10⁶ cells were treated with different reagents as indicated and lysed in RIPA buffer (50 mM Tris HCl, 137 mM NaCl, 0.5% Na Deoxycholate, 1% Triton X-100, 0.1% SDS, protease inhibitors). Proteins were separated by SDS-PAGE and transferred to a nitrocellulose membrane (Amersham Biosciences, Little Chalfon, UK) using a semi-dry blotting approach. The following antibodies were used: p53 (DO-1) antibody was purchased from Santa Cruz Biotechnology (Heidelberg, Germany). Tubulin and actin (A5441) antibodies were purchased from Sigma-Aldrich (St. Louis, USA).

### Immunoprecipitation and Metabolic Pulse-Labeling Experiments

For immunoprecipitation, cells were treated for 4 h with Etoposide and/or Roc-A in the absence or presence of 100 nM Bortezomib (Enzo Life Sciences, Lörrach, Germany) In the case of metabolic pulse-labeling experiments, treatment was followed by adding 100 μCi/ml of ³⁵5-methionine-labeling mix (PerkinElmer, Waltham, Mass., USA) to the medium for 0-15 min. Subsequently, cells were washed in ice-cold PBS, lysed in IP buffer (20 mM Tris-HCl, 5 M NaCl, 2 mM EDTA, 1% Triton X-100, protease inhibitors) and centrifuged (10,000 g, 20 min) to clear lysates. Aliquots were taken for input control and lysates were incubated overnight with sepharose-coupled protein A beads, anti-p53 antibody (FL-393; Santa Cruz, Heidelberg, Germany) or isotype control antibody (Sigma-Aldrich, Munich, Germany). Two wash-steps with IP buffer preceded boiling of beads in denaturing sample buffer at 95° C. for 5 mM. Incorporation of ³⁵5-methionine into p53 protein was detected by the phosphoimaging system FLA-7000 IR (Fujifilm Europe GmbH, Düsseldorf, Germany).

### Translation Assay

The relative amount of protein synthesis was determined by measuring the amount of incorporation of ³⁵S-methionine into the protein. Briefly, cells were pre-cultured in methionine-free medium (supplemented with 10% dialyzed FCS) for 3 h, followed by incubation with 3.5 μCi of ³⁵S-methionine-labeling mix (PerkinElmer, Waltham, Mass., USA) per 8×10⁵ cells for 6 h as indicated. After the treatment, cells were washed twice with ice-cold PBS and lysed in RIPA buffer. 50 μl of each lysate were added to 1 ml of Liquid Scintillation Cocktail solution (Beckman Coulter, Brea, Calif., USA) and the amount of incorporated radioactivity was determined by liquid scintillation counting.

### Isolation of Primary Murine Splenocytes

P53^{-/-} C57B1/6 mice (B6.Trp53tm1Tyj) were kindly provided by Liu H-K (German Cancer Research Center, Heidelberg, Germany). Spleens of 8-12 week old p53^{-/-}, and p53^{+/+} mice were isolated in parallel, minced and incubated for 30 min in RPMI-1640 medium supplemented with DNase I (50 U/ml) and Collagenase IV (1 mg/ml) at 37° C. and 5% CO₂. Splenocytes were filtered by 40 μM cell strainer, washed twice with ice-cold wash buffer (PBS, 0.5% FCS, 2 mM EDTA) and resuspended in Oxford medium (RPMI 1640, 10% FCS, 100 μg/ml Penicillin, 100 μg/ml Streptamycin, 10 mM Hepes, 50 μM β-Mercaptoethanol, 2 mM L-glutamine, 1 mM sodium pyruvate, 100 μM non-essential amino acids) at a concentration of 2×10⁶ cells/ml.

### Enrichment of Hematopoietic Stem and Progenitor (HSPCs) Cells by Lineage Depletion

For enrichment of HSPCs, 8 week old C57B1/6 wild-type mice (Harlan Laboratories, Rolβdorf, Germany) were sacrificed and bone marrow was prepared from hind legs (femur and tibia), fore legs (humerus), hips (ilium), and vertebral column (columna vertebralis) by crushing bones in RPMI-1640 medium (Sigma-Aldrich, Munich, Germany) supplemented with 2% FCS. To perform lineage depletion, bone marrow cells were incubated on ice for 40 minutes with rat monoclonal antibodies against common epitopes expressed on mature blood and bone marrow cells (CD11b (M1/70), Gr-1 (RB6.8C5), CD4 (GK1.5), CD8a (53.6.7), Ter119 (Ter119) and B220 (RA3-6B2)). Subsequently, cells were washed and incubated for 15 minutes on ice with anti-rat IgG-coated Dynabeads (4.5 μm supermagnetic polystyrene beads, Invitrogen), 1 ml of beads per 3×10⁸ bone marrow cells. Cells expressing lineage markers were depleted using a magnet and the remaining lineage-negative cells were isolated and washed. To provide optimal conditions for HSPCs in downstream experiments lineage-negative hematopoietic stem and progenitor-enriched cells were cultured in StemPro®-34 serum-free medium (Invitrogen, Darmstadt, Germany) supplemented with nutrient supplement (Invitrogen, Darmstadt, Germany) as well as recombinant TPO (50 ng/ml, (Peprotech, Hamburg, Germany)), SCF (50 ng/ml, (Peprotech, Hamburg, Germany)) and Flt3-ligand (50 ng/ml, (Peprotech, Hamburg, Germany)).

### Knockdown Experiments

siRNAs specific for p53 mRNA was 5′-GUAAUCUACUGGGACGGAAtt-3′ (SEQ ID NO: 1; [Applied Biosystems, Darmstadt, Germany]). 1.5×10⁷ human peripheral blood T cells were transfected with 2 μM of p53 siRNA or of scrambled siRNA (Qiagen, Hilden, Germany) using Amaxa Human T Cell Nucleofector Kit (Lonza, Basel, Switzerland) according to the manufacturer's instructions. The Amaxa Nucleofector program U-014 was used for transfection.

### Determination of DNA Damage

DNA damage was determined by quantification of γ-H2AX foci formation and by alkaline single-cell gel electrophoresis assay (comet assay). For γ-H2AX staining, cells were treated as indicated, fixed in 3% formaldehyde and permeabilized in 90% methanol. Following storage at −20° C. overnight, cells were incubated with mouse serum to block unspecific binding and stained with antibody directed against γ-H2AX (AlexaFluor 488-coupled, 2F3 [BioLegend, Fell, Germany]), or with isotype control antibody (AlexaFluor 488-coupled [BioLegend, Fell, Germany]). The amount of γ-H2AX foci formation was determined by FACS measurement. Cell aliquots were taken and confocal microscopy was carried out to visualize Etoposide-induced γ-H2AX foci formation. Nuclei were stained with DAPI mounting medium (Dianova, Hamburg, Germany). Comet assays were carried out as previously described (Greve et al., PloS one. 2012; 7: e47185). Briefly, electrophoresis of cellular genomic DNA was performed under alkaline conditions at 4° C. The amount of DNA damage was measured by “Olive Tail Moment”. Analysis of cellular DNA damage was carried out by fluorescence microscopy, using a fully automated cell scanning system Metafer-4 (Metasystems, Altlutβheim, Germany).

### EXAMPLE 2

### Roc-A Protects Non-Malignant Cells Against DNA Damage-Induced Cytotoxicity

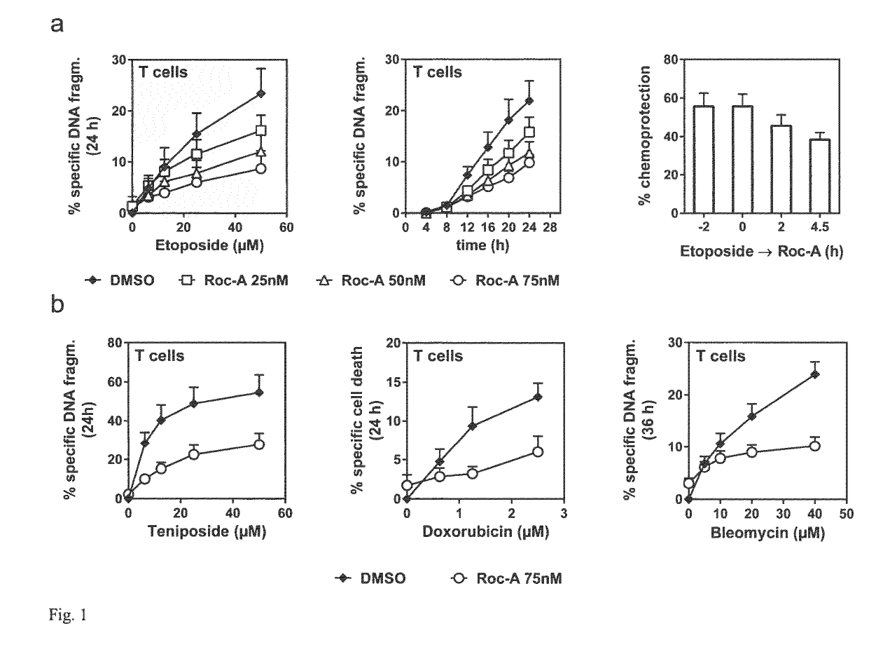

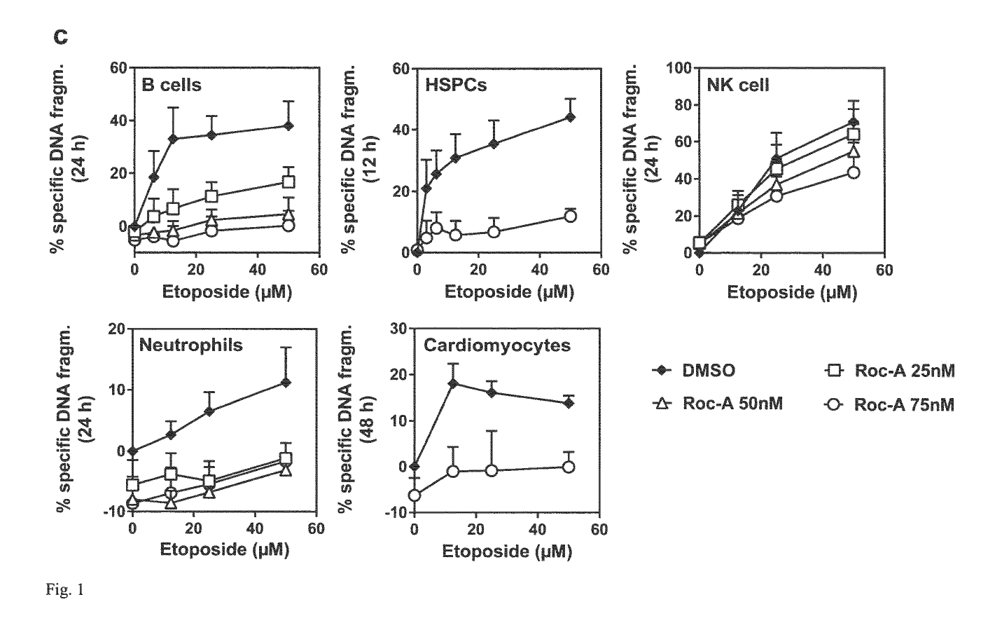

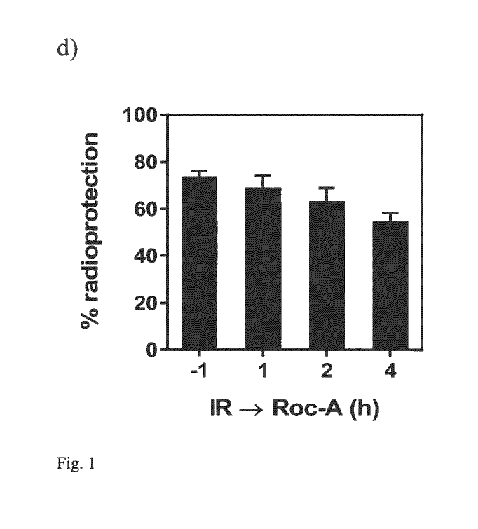

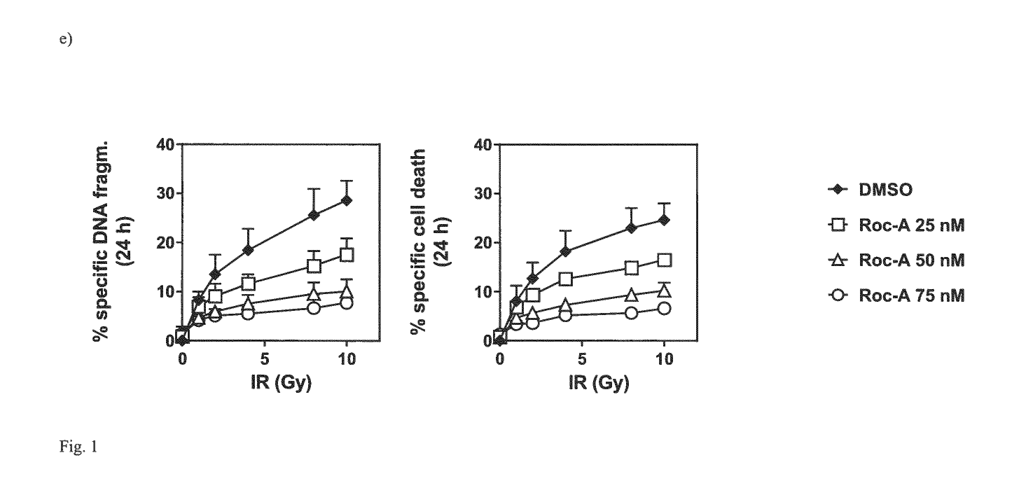

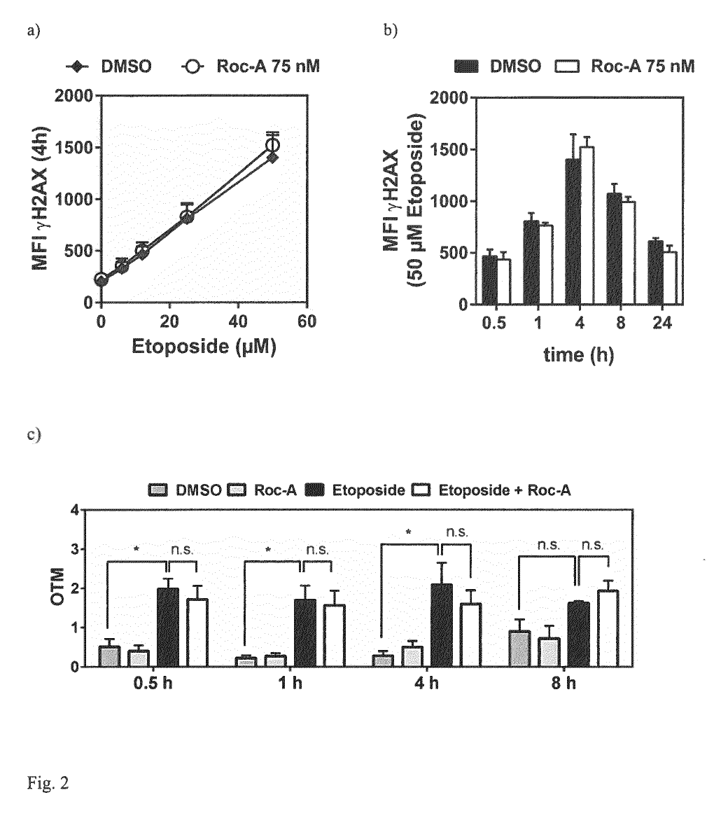

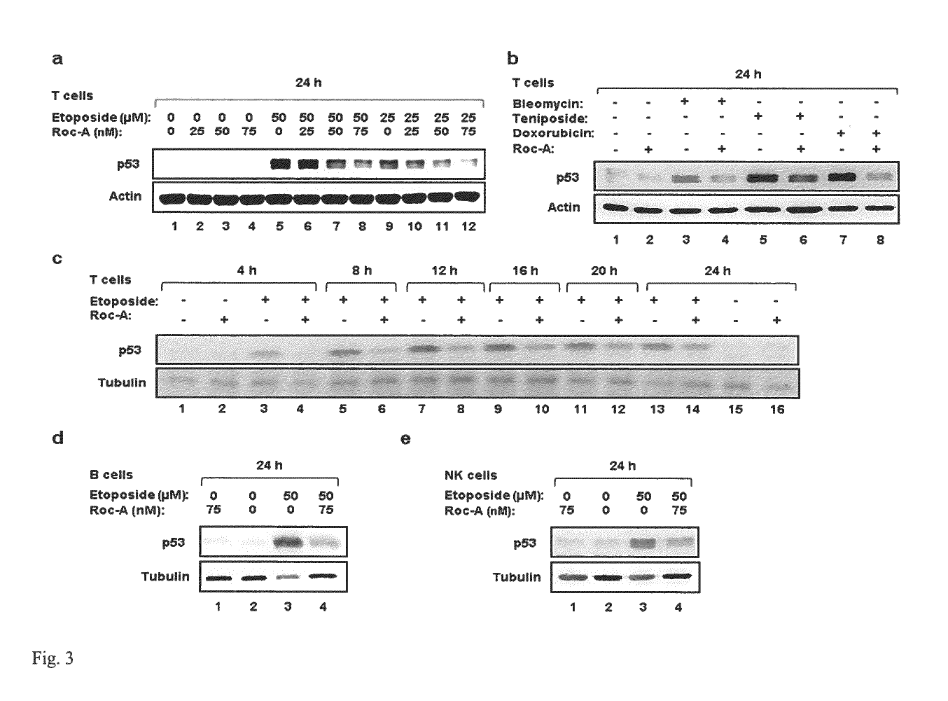

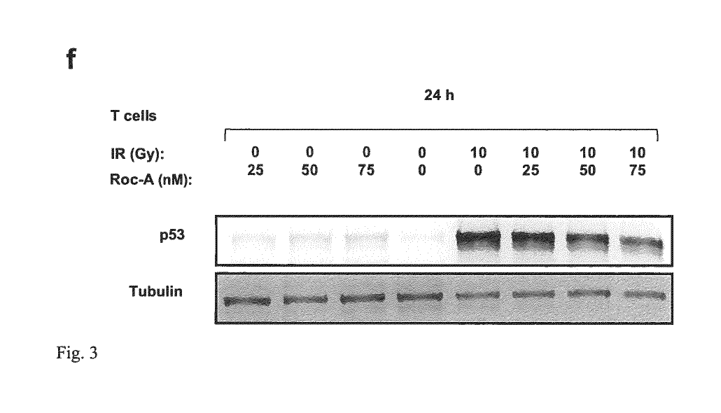

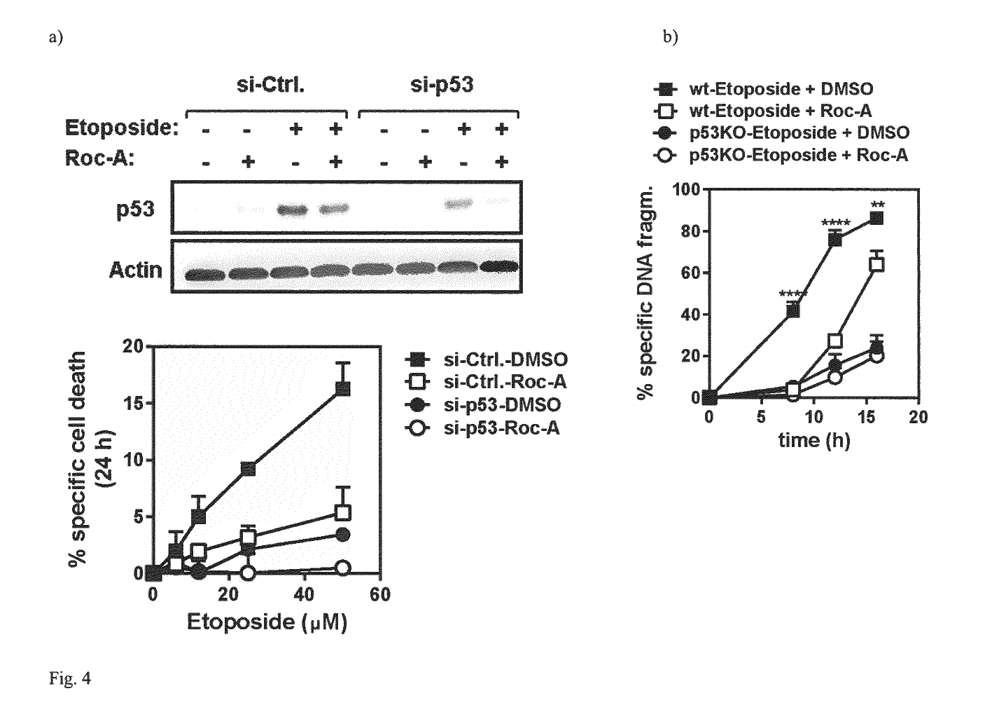

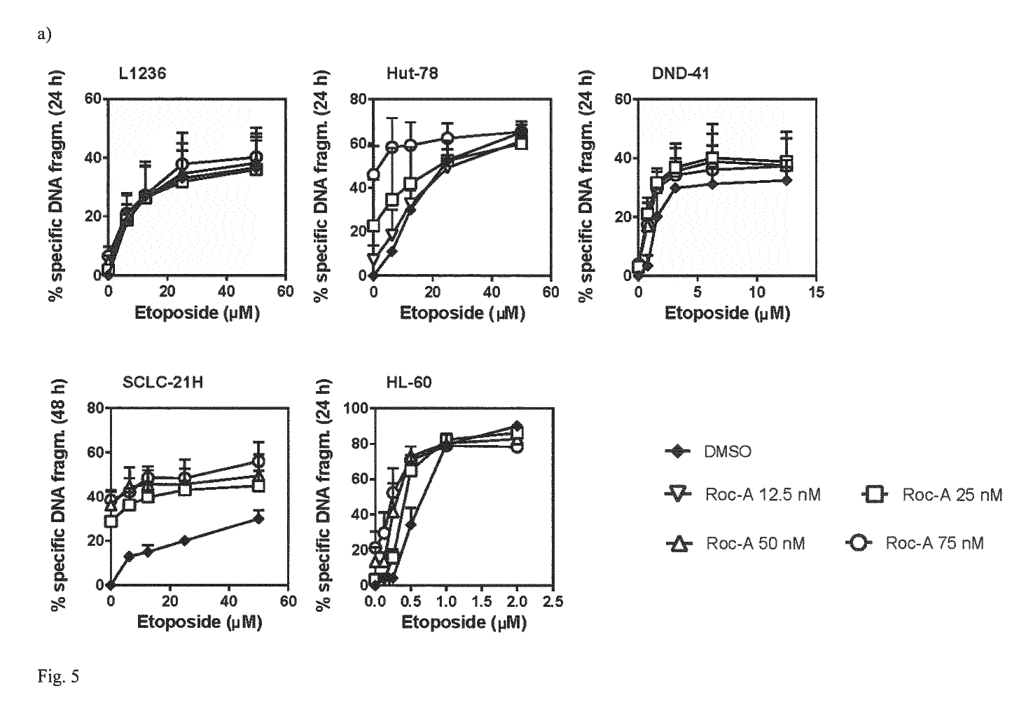

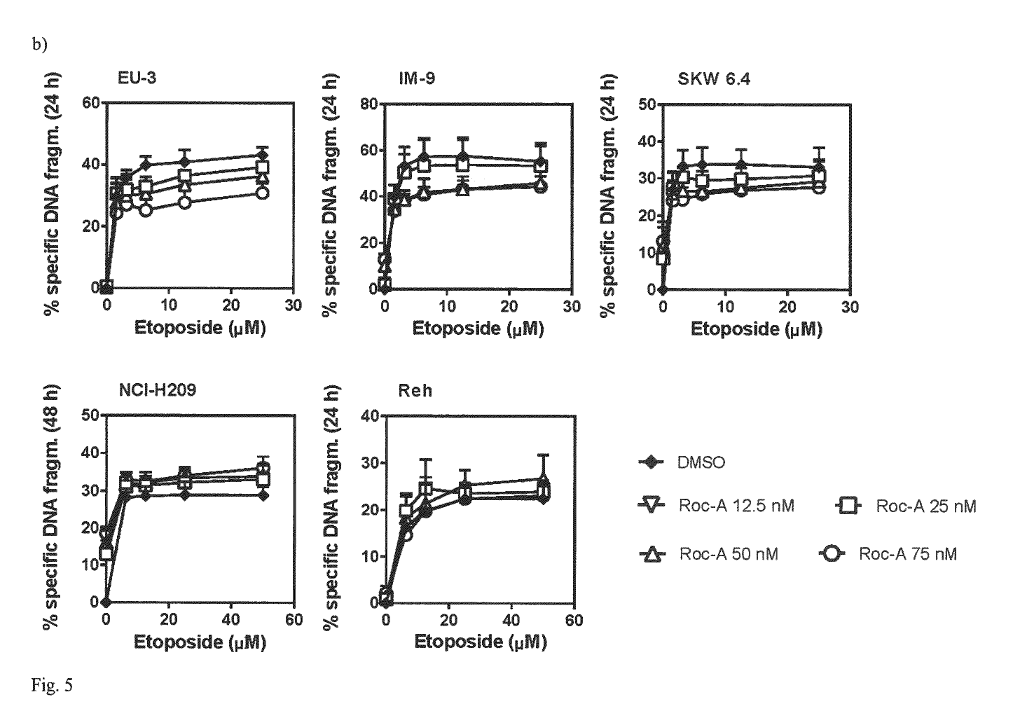

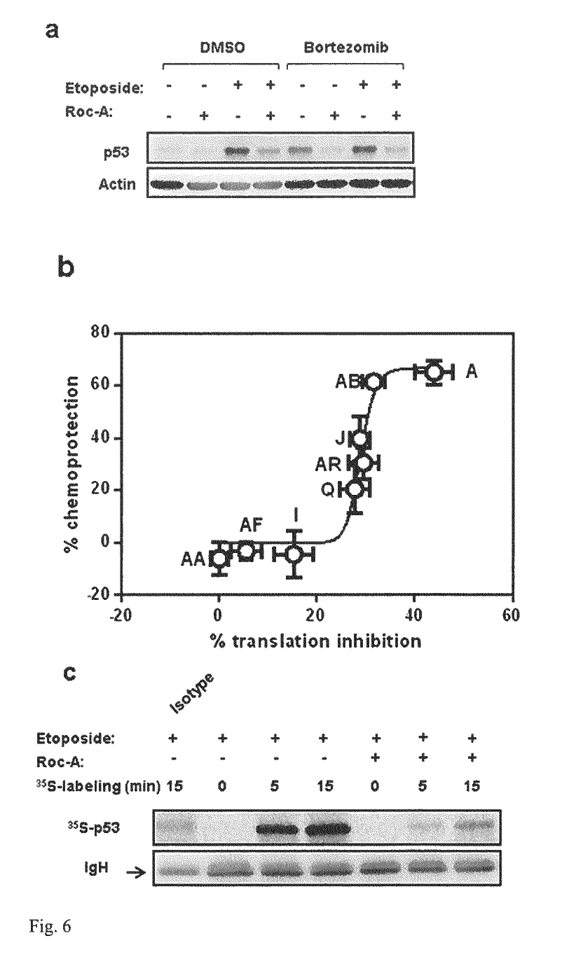

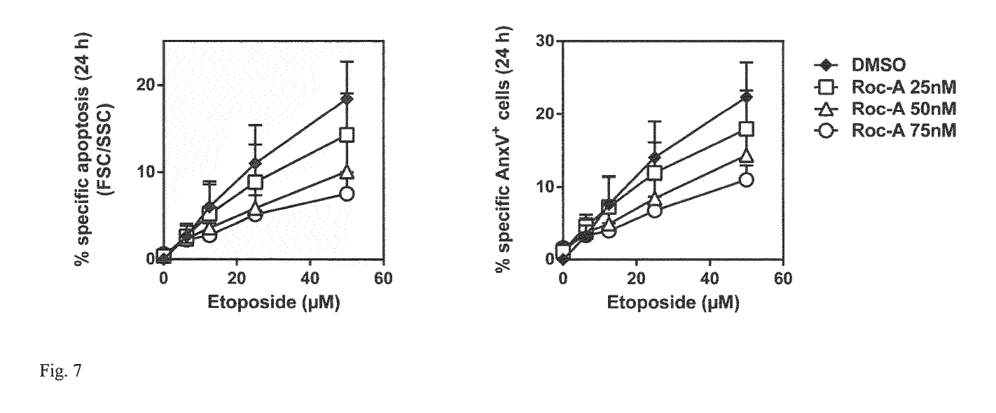

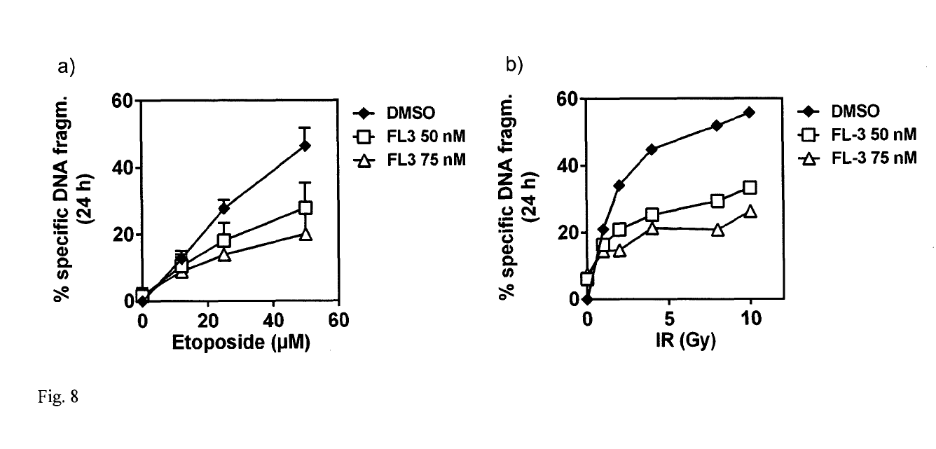

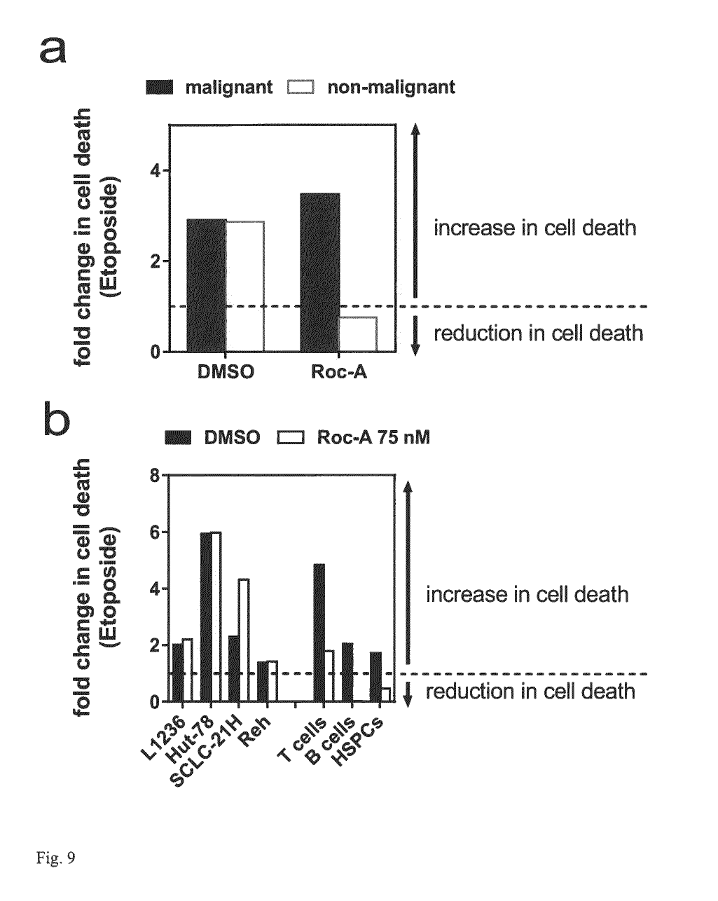

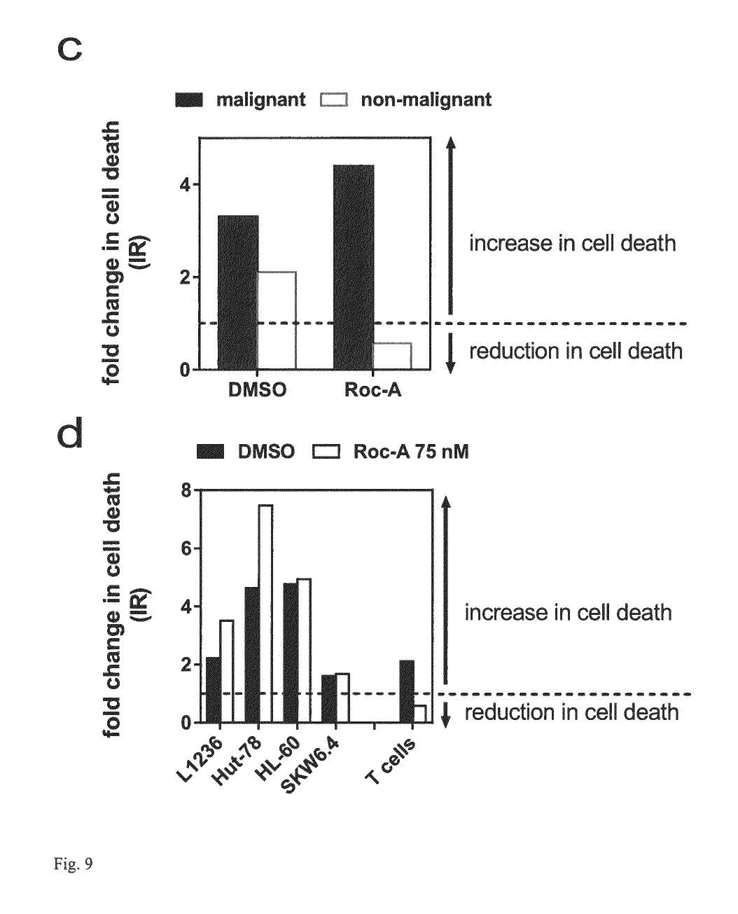

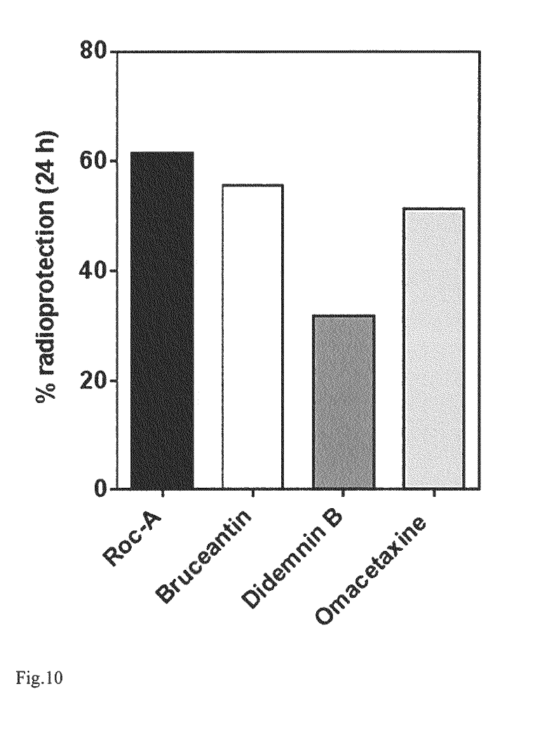

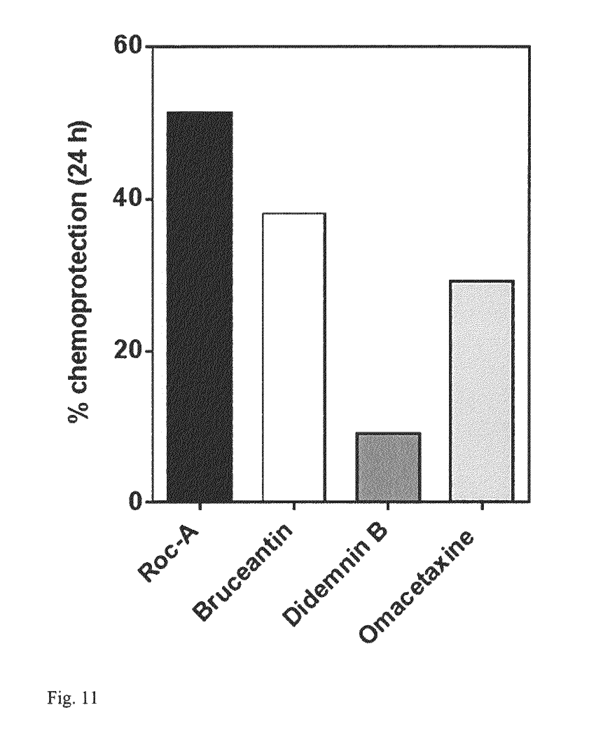

We treated normal T cells with increasing concentrations of Etoposide with or without different concentrations of Roc-A. After 24 h treatment, apoptotic cell death was measured by specific DNA fragmentation, Annexin V staining or determination of apoptotic-like changes in cell size and cellular granularity (FSC/SSC profile). The experiments showed that Etoposide treatment caused cell death of normal T cells, which was reduced in the presence of Roc-A in a dose-dependent manner to more than 50% (FIG. 1a left panel and FIG. 7). Kinetic analysis showed that Roc-A could reduce the toxicity of Etoposide at all measured time points (FIG. 1a, middle panel). Strikingly, the chemo-protective effect could be even seen when Roc-A was administered after several hours of Etoposide treatment (FIG. 1a right panel).

To investigate whether Roc-A could also protect normal cells from cell death induced by other DNA damaging anti-cancer drugs, we treated T cells with increasing doses of Teniposide, Doxorubicin and Bleomycin in the presence or absence of Roc-A. The experiment showed that Roc-A could reduce drug-induced apoptosis in all cases (FIG. 1b). Moreover, Roc-A reduced ionizing radiation-induced apoptosis in T cells (FIG. 1e). The radioprotective effect mediated by Roc-A was the highest when cells were treated with Roc-A 1 h before radiation of cells (FIG. 1d). However, when cells were treated 4 h after radiation, the radioprotective effect was still higher than 50% (FIG. 1d).

Next, we asked whether Roc-A could also protect other normal primary cells from DNA damage-induced toxicity. To address this question, we examined the protective effect of Roc-A on Etoposide-treated human peripheral blood B cells, NK cells, neutrophils, cardiomyocytes and murine hematopoietic stem and progenitor cells (HSPCs). The experiments revealed that all examined cells were protected by Roc-A against Etoposide-induced apoptosis (FIG. 1c). Taken together, these data indicate that Roc-A can protect primary non-malignant cells from DNA damage-induced cytotoxicity. Similar data were obtained for FL3 (FIG. 8).

### EXAMPLE 3

### Roc-A Exerts its Protection Downstream of DNA Damage

Genotoxins such as Etoposide induce apoptosis mainly through induction of DNA damage (Roos & Kaina, Trends Mol Med. 2006; 12: 440-450). We therefore asked whether Roc-A could prevent genotoxin-induced DNA damage and thereby reduce genotoxin-induced cell death. To address this question, we determined the level of the DNA-damage marker γ-H2AX, which is generated around the site of a DNA double-strand break (Rogakou et al., J Biol Chem. 1998; 273: 5858-5868). Etoposide treatment resulted in an increase in γ-H2AX foci formation in a concentration- and time-dependent manner (FIGS. 2a and b). A maximum amount of γ-H2AX foci formation was observed at 4 h post-treatment (FIG. 2b). However, Roc-A did not block Etoposide-induced γ-H2AX foci formation (FIG. 2a-b). Similar results were obtained by alkaline single-cell gel electrophoresis assay (Comet assay) which detects both DNA single- and double-strand breaks (FIG. 2c). Therefore, the protective effect of Roc-A appears to be downstream of DNA damage.

### EXAMPLE 4

### Roc-A Inhibits DNA Damage-Induced Increase in p53 Expression

The transcription factor p53 is a major regulator of DNA damage-induced apoptosis (Lowe et al., Cell. 1993; 74: 957-967). Therefore, we investigated the effect of Roc-A on the expression level of p53. T cells were treated with increasing concentrations of Etoposide in the presence or absence of Roc-A and p53 protein expression was analyzed by immunoblot. The experiment showed that Etoposide treatment increased p53 protein levels. However, in the presence of Roc-A, p53 expression was blocked in a dose-dependent manner (FIG. 3a). Roc-A-mediated suppression of p53 upregulation was not specific for Etoposide, as inhibition was also observed in ionizing radiation (IR)-, Bleomycin-, Teniposide- and Doxorubicin-treated cells (FIG. 3b, f). Kinetic analysis showed that the increase in p53 protein levels could be detected as early as 4 h after Etoposide treatment (FIG. 3c). Roc-A could inhibit upregulation of p53 at all time-points analyzed (FIG. 3c). The time of p53 upregulation coincided with the onset of apoptosis induction (FIG. 1b). To ensure that Roc-A-mediated suppression of p53 upregulation was not only specific for T cells, the effect of Roc-A on p53 expression was also examined in B and NK cells treated with Etoposide. Consistent with the results obtained from T cells, Etoposide-induced p53 increase in B and NK cells was also suppressed by Roc-A (FIGS. 3d and e). These data indicate that Roc-A might protect normal tissue from DNA damage-induced apoptosis by down-regulation of genotoxin-induced p53 expression.

### EXAMPLE 5

### p53 Plays an Essential Role in Roc-A Mediated Protection

To further investigate the role of p53 in Roc-A-mediated protection of T cells against DNA damage-induced apoptosis, we performed a p53 knock-down experiment using a siRNA approach (FIG. 4a, upper panel). The experiment showed that down-regulation of p53 protein levels in T cells rendered the cells more resistant to Etoposide and reduced cell death to a level similar to Roc-A treatment (FIG. 4a, lower panel). However, Roc-A could still further down-regulate Etoposide-induced apoptosis in p53-knockdown cells which most likely occurred due to inefficient knockdown of p53 (FIG. 4a, upper panel). Therefore, we examined the effect of Roc-A on p53 knock-out (KO) cells. Splenocytes derived from wild-type (WT) and p53-KO mice were treated with Etoposide in the presence or absence of Roc-A. In line with the data observed in primary human cells, Roc-A protected splenocytes derived from p53-WT but not from p53-KO animals against Etoposide-induced cell death (FIG. 4b). These results demonstrate that p53 plays an essential role in Roc-A-mediated protection.

It is notable that a number of studies show that transient pharmacological or genetic inactivation of p53 before or after genotoxic stress does not lead to increased carcinogenesis (Christophorou et al., Nature. 2006; 443: 214-217; Hinkal et al., PloS one. 2009; 4: e6654; Komarov, 1999; 285: 1733-1737). Moreover, recent publications indicate that the tumor suppressor function of p53 is independent from its functions on apoptosis and cell cycle (Liet al., Cell. 2012; 149: 1269-1283; Brady et al., Cell. 2011; 145: 571-583).

### EXAMPLE 6

### Roc-A does not Protect Malignant Cells with Non-Functional p53

Since Roc-A-mediated protection of non-malignant cells from DNA-damage-induced cell death is largely p53-dependent, we predicted that Roc-A would not protect cancer cells which have non-functional p53. As expected, Roc-A did neither protect p53 mutated (L1236 (Feuerborn et al., Leuk Lymphoma. 2006; 47: 1932-1940), Hut-78 (Cheng & Haas, Mol Cell Biol. 1990; 10: 5502-5509), DND-41 (Zhu et al., Cell Death Differ. 2009; 16: 1289-1299), SCLC-21H (Forbes et al., Br J Cancer. 2006; 94: 318-322) nor p53-deficient (HL-60 (ibid.) cancer cell lines against Etoposide-induced cell death (FIG. 5a). We then further asked whether Roc-A would protect tumor cells which have functional p53. To investigate this question, we tested cancer cell lines EU-3 (Zhou et al., Leukemia. 1998; 12: 1756-1763), IM-9 (Jia et al., Mol Carcinog. 1997; 19: 243-253), Reh (Zhou et al, loc cit.), SKW6.4 (Barbarotto et al., J Cell Biochem. 2008; 104: 595-605) and NCI-H209 (Fujita et al. Int J Oncol. 1999; 15: 927-934) which have a WT p53 protein. The experiments showed that EU-3, SKW6.4 and IM-9 cells were shown to be protected by Roc-A from Etoposide-induced cell death but to a lesser extent than non-malignant cells (FIG. 5b).

### EXAMPLE 7

### Roc-A Suppresses p53 Upregulation Via Inhibition of Protein Synthesis

p53 protein expression can be regulated at the level of transcription, translation, and ubiquitination-mediated degradation (Marine & Lozano, Cell Death Differ. 2010; 17: 93-102). It has been shown that upon DNA damage p53 undergoes post-translational modifications leading to its deubiquitination and, thus, stabilization (Lee & Gu, Cell Death Differ. 2010; 17: 86-92). To investigate whether Roc-A could decrease p53 stability, we treated T cells with the proteasome inhibitor Bortezomib to block proteasome-mediated degradation. Bortezomib treatment led to an increase in p53 in the absence of Roc-A (FIG. 6a). However, Bortezomib could not increase p53 in the presence of Roc-A and did also not interfere with the ability of Roc-A to block Etoposide-induced p53 upregulation (FIG. 6a).

p53 has also been shown to be upregulated at the translational level following DNA damage (Takagi et al., Cell. 2005; 123: 49-63, Gajjar et al., Cancer cell. 2012; 21: 25-35). Roc-A has been well documented to inhibit protein translation (Polier et al., Chem Biol. 2012; 19: 1093-1104; Sadlish et al., ACS Chem Biol. 2013; doi:10.1021/cb400158t; Bleumink et al., Cell Death Differ. 2011; 18: 362-370; Cencic et al., PloS one. 2009; 4: e5223). Thus, we expected that Roc-A-mediated suppression of genotoxin-induced p53 upregulation may be regulated by inhibition of p53 protein synthesis. To test this, we examined the effects of different Roc-A derivatives which have been shown to exert different activities on inhibition of ERK-mediated protein synthesis (Polier et al., op. cit.). By means of [³⁵S]methionine incorporation analysis, Roc-A, AB, J, AR, and Q, which have been shown to inhibit ERK activation with different efficacies (Polier et al., op. cit.), inhibited [³⁵S]methionine incorporation at different degrees which correlated with different levels of protection of normal T cells from Etoposide-induced cell death (FIG. 6b). In contrast, Roc-AA, AF and I, which do not show any or very little inhibitory effects on ERK activity (Polier et al., op. cit.), did not inhibit protein translation and did not protect T cells against Etoposide-induced cytotoxicity (FIG. 6b).

To further confirm that Roc-A inhibits p53 protein synthesis, we carried out a [³⁵S]methionine-metabolic pulse-labeling experiment and then immunoprecipitated p53 after Etoposide treatment. The experiment showed that Roc-A suppressed [³⁵S]methionine incorporation into the p53 protein (FIG. 6c). Thus, Roc-A suppresses DNA-damage-induced upregulation of p53 at the translational level.

### EXAMPLE 8

### Roc-A Enables the Use of High-Dose Chemo/Radiotherapy by Protecting Healthy Cells from DNA-Damage Induced Cell Death

Roc-A specifically prevents the cause of chemotherapeutic and radiation-induced side-effects, i.e., the death of healthy cells. Roc-A does not protect p53-deficient/mutated cancers and protects p53 proficient tumors at least to a lesser extent as compared to healthy cells. Hence, Roc-A broadens the therapeutic window of chemotherapeutics and radiation which allows for higher radiation or drug dosage in tumor patients (FIG. 9). An increase in the dose of Etoposide from 6.25 μM to 50 μM leads to an approximately 3-fold increase in Etoposide-induced cell death in malignant cells (FIG. 9a). However, increased doses of Etoposide also increase cell death of non-malignant cells up to 3-fold (FIG. 9a). Consequently, high-dose therapy is not possible, as side effects would be too high. When cells were treated with Roc-A in parallel to Etoposide, an increase in the doses of Etoposide only resulted in increased cell death in malignant cells. Similar results were obtained for ionizing radiation (FIG. 9c, d). Hence, Roc-A enables the safe use of high-dose chemo/radiotherapy.

1-19. (canceled)

20. A method of preventing in a subject requiring high-dose chemotherapy and/or high-dose radiotherapy adverse events caused by said therapy or therapies, comprising
a) administering an effective dose of an inhibitor of protein translation to said subject,
b) thereby preventing in a subject requiring high-dose chemotherapy and/or high-dose radiotherapy adverse events caused by said therapy or therapies.

21. The method of claim 20, wherein the disease requiring high-dose chemotherapy and/or high-dose radiotherapy is cancer.

22. The method of claim 20, wherein the inhibitor of protein translation is a flavagline of the formula (I) 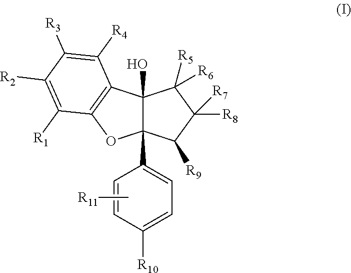
wherein
R1 is selected from —H, halogen, and alkyl;
R2 is selected from optionally substituted alkoxy, halogen, and alkyl;
R3 is selected from —H, halogen, and alkyl;
or R2 and R3 together form a —O(CH2)nO— unit, with n=1 or 2;
R4 is selected from alkoxy, halogen, —H, and alkyl;
R5 is selected from hydroxyl, acyloxy, —H, and amino;
R6 is selected from —H, halogen, alkyl, and amino;
or R5 and R6 together form an oxo or hydroxyimino group;
R7 is —H;
R8 is selected from —CONR16R17, —H, and —COOR15, wherein
R15 and R16 are independently selected from methyl and —H, and
R17 is selected from methyl, —H, 4-hydroxybutyl, and 2-tetrahydrofuryl;
R9 is selected from optionally substituted phenyl and optionally substituted hetaryl;
R10 is selected from alkoxy, —H, halogen, and alkyl; and
R11 is selected from —H, hydroxyl, halogen, alkoxy, and alkyl;
or R10 and R11 are in ortho-position to each other and together form a —O(CH2)ₙO— unit, with n=1 or 2.

23. The method of claim 22, wherein in the inhibitor of protein translation of formula (I):
R2 is selected from the group consisting of methoxy and a group —O—(CH2)n-R18 wherein n is 1, 2, 3, or 4 and R18 is hydroxyl, —NMe2, —OCONMe2, —OCONH2 or morpholine;
R5 is a substituted amino selected from the group consisting of monoalkylamino, dialkylamino, —NHCHO, —NHSO2Me, —NHAc, —NHCOEt, —NHCOCH2OH, —NHCOCH2NMe2, —NHCONMe2, —NHCONH2, and —NHCOOMe; and —NR12-CHR13-COOR14, with
R12 being selected from —H and alkyl;
R13 being selected from phenyl and benzyl, which both may carry a substituent from the group hydroxyl, indolyl and imidazolylmethyl, and alkyl which may be substituted by a group selected from —OH, —SH, alkoxy, thioalkoxy, amino, monoalkylamino, dialkylamino, carboxy, carboxyalkyl, carboxamide and guanidino groups;
or R12 and R13 together form a —(CH2)3- or —(CH2)4-group;
R14 being selected from alkyl and benzyl, in which case R6 is hydrogen;
R6 is a substituted amino selected from the group consisting of —NHCHO, —NHSO2Me, —NHAc, —NHCOEt, —NHCOCH2OH, —NHCOCH2NMe2, —NHCONMe2, —NHCONH2, and —NHCOOMe;
R9 is a alkoxy- or halogen-substituted phenyl; and
R10 is —Br.

24. The method of claim 20, wherein the inhibitor of protein translation is a Rocaglamide and wherein the inhibitor of protein translation is not Rocaglamide AA (C-1-O-acetyl-methylrocaglate), Rocaglamide AF (30,40-methylendioxy-methylrocaglate) or Rocaglamide I (C-1-O-acetyl-30-hydroxy-rocaglamide).

25. The method of claim 20, wherein the inhibitor of protein translation is Rocaglamide Q (demethylrocaglamide); Rocaglamide AR (1-oxo-40-demethoxy-30, 40-methylenedioxyrocaglaol); Rocaglamide J (30-hydroxyaglafoline); or a derivative thereof.

26. The method of claim 20, wherein the inhibitor of protein translation is Rocaglamide AB (1-O-acetyl-rocaglamide), racemic bromo-demethoxy-rocaglaol (FL3), or a derivative thereof.

27. The method of claim 20, wherein the inhibitor of protein translation is (1R,2R,3S,3aR,8bS)-1,8b-dihydroxy-6,8-dimethoxy-3a-(4-methoxyphenyl)-N,N-dimethyl-3-phenyl-2,3-dihydro-1H-cyclopenta[b][1]benzofuran-2-carboxamide (Rocaglamide A) or a derivative thereof.

28. The method of claim 20, wherein high-dose chemotherapy is high dose therapy with an agent selected from the list consisting of etoposide, bleomycin, doxorubicin, teniposide.

29. The method of claim 20, wherein the adverse effects are adverse effects of the blood system.

30. The method of claim 20, wherein the adverse effects are adverse effects caused by a diminished number of at least one kind of blood cell.

31. The method of claim 20, wherein the adverse effects are adverse effects caused by a diminished number of T-cells, B-cells, NK cells, and/or neutrophils.

32. The method of claim 20, wherein the adverse effects are adverse effects caused by a diminished number of hematopoietic stem and progenitor cells.

33. A combined preparation for simultaneous, separate, or sequential use comprising at least one inhibitor of protein translation or a pharmaceutically acceptable salt thereof; and at least one chemotherapeutic agent for use in high-dose chemotherapy of disease.

34. A kit comprising at least one inhibitor of protein translation and instructions on administering high-dose chemotherapy and/or instructions on administering high-dose radiotherapy in the presence of said inhibitor of protein translation.


SEQUENCE LISTING (1 sequence)

SEQ ID NO: 1 - DNA, 21 bp, artificial sequence
  knockdown oligonucleotide
guaaucuacugggacggaatt